(19) United States
(12) Patent Application Publication
(10) Pub. No.: US 2022/0401073 A1
(43) Pub. Date: Dec. 22, 2022

(54) SYSTEMS AND METHODS FOR HARMONIC MOTION ELASTOGRAPHY

(71) Applicant: THE TRUSTEES OF COLUMBIA UNIVERSITY IN THE CITY OF NEW YORK, New York, NY (US)

(72) Inventors: Elisa E. KONOFAGOU, New York, NY (US); Alireza NABAVIZADEHRAFSANJANI, Ann Arbor, MI (US)

(73) Assignee: THE TRUSTEES OF COLUMBIA UNIVERSITY IN THE CITY OF NEW YORK, New York, NY (US)

(21) Appl. No.: 17/738,410

(22) Filed: May 6, 2022

Related U.S. Application Data
(63) Continuation of application No. PCT/US2020/059715, filed on Nov. 9, 2020.
(60) Provisional application No. 62/932,293, filed on Nov. 7, 2019.

(51) Int. Cl. A61B 8/08

(52) U.S. Cl. CPC A61B 8/48; A61B 8/5223; A61B 8/08

(57) ABSTRACT

The present subject matter relates to techniques for harmonic motion elastography. The disclosed system can include a focused ultrasound (FUS) transducer for applying a push to a target tissue; an imaging transducer for obtaining radio frequency (RF) signals from the target tissue, and a processor configured to estimate the mechanical properties of the target tissue by extracting a shear wave from the RF signals and estimating a shear wave speed.

### CROSS-REFERENCE TO RELATED APPLICATION

This application is a continuation of International Patent Application No. PCT/US2020/059715 filed Nov. 9, 2020, which claims priority to U.S. Provisional Patent Application No. 62/932,293, which was filed on Nov. 7, 2019, the entire contents of which are incorporated by reference herein.

### GRANT INFORMATION

This invention was made with government support under grant numbers R01-CA228275 awarded by the National Institutes of Health. The government has certain rights in the invention.

### BACKGROUND

Certain ultrasound-based elastography techniques can be used for the mechanical evaluation of soft tissues. For example, Magnetic Resonance Elastography (MRE) methods can be used on various organs like the brain, liver, heart, and muscle to evaluate their mechanical properties. Shear wave ultrasound elastography is a type of dynamic elastography, in which the mechanical properties of the tissues can be estimated by using radiation force to introduce shear waves and measuring the shear and Young's moduli by tracking the generated shear waves. Harmonic motion imaging is an ultrasound-based elastography technique for measuring Young's moduli.

However, these techniques can fail to provide a quantitative measurement of the mechanical properties of tissues. Without such quantification, identifying a lesion area and a non-lesion area within the tissue can be challenging. For example, radiologically, it can be challenging to accurately delineate pancreatic ductal adenocarcinoma (PDA) tumor's margin, as these ultrasound-techniques can provide limited specificity for differentiating tumor part from desmoplasia. Due to these limitations, surgeons can have difficulty to make operational decisions because they do not know where exactly the tumor ends.

Therefore, there is a need for improved techniques for delineating cancer/tumor parts from healthy tissue.

### SUMMARY

The disclosed subject matter provides techniques for harmonic motion elastography. The disclosed subject matter provides systems and methods for measuring a mechanical property of target tissue.

In certain embodiments, a system for harmonic motion elastography can include a focused ultrasound (FUS) transducer, an imaging transducer, and a processor. The FUS transducer can generate an oscillatory motion of a target tissue by applying a push to the target tissue. The imaging transducer can obtain radio frequency (RF) signals from the oscillatory motion during the application of the push. The processor can estimate the mechanical properties of the target tissue by extracting a shear wave from the RF signals obtained using the imaging transducer and estimating a shear wave speed based on the extracted shear wave. In non-limiting embodiments, the mechanical property can include elasticity, stiffness, viscosity, poroelasticity, or combinations thereof. In some embodiments, the push can generate the deformation of the target tissue. In some embodiments, the system can be configured to generate a mechanical property map with a single push.

In certain embodiments, the FUS transducer can move in a raster scanning manner. In non-limiting embodiments, the imaging transducer can obtain radio frequency data in real-time.

In certain embodiments, the processor can conduct beamforming on the RF signal and/or generate a mechanical property map of the target tissue through a 1D cross-correlation. In non-limiting embodiments, the processor can generate a mechanical property map. In some embodiments, the processor can identify a boundary between a lesion area and a non-lesion area. In non-limiting embodiments, the processor can be implemented in a graphical processing unit.

In certain embodiments, the disclosed subject matter provides methods for measuring a mechanical property of target tissue. An example method can include modulating the target tissue by inducing a push with a focused ultrasound (FUS) ultrasound, obtaining radio frequency (RF) signals from the target tissue using an imaging transducer, and estimating a mechanical property based on the RF signals. In non-limiting embodiments, the mechanical property can include elasticity, stiffness, viscosity, poroelasticity, or combinations thereof.

In certain embodiments, the method can further include conducting beamforming on the RF signals and estimating RF displacement of the target tissue through a 1D cross-correlation.

In certain embodiments, the RF signals can be obtained through a single push. In non-limiting embodiments, the method can further include adjusting the frequency of the push depending on the target tissue. In some embodiments, the method can further include moving the focused ultrasound in a raster scanning manner.

In certain embodiments, the method can include identifying a boundary between a lesion area and a non-lesion area of the target tissue. In non-limiting embodiments, the target tissue can be a pancreatic ductal adenocarcinoma tumor.

In certain embodiments, the method can further include extracting shear wave from the estimated RF displacement and estimating shear wave speed. In non-limiting embodiments, the method can further include calculating Young's modulus using the estimated shear wave speed and generating a Young's modulus map.

The disclosed subject matter will be further described below.

### BRIEF DESCRIPTION OF THE DRAWINGS

FIG. 1 provides a diagram of an example system in accordance with the disclosed subject matter.

FIGS. 2A-21 provides images showing different fibrosis stages using H&E stained slides and HMI displacement, and Young's modulus maps overlaid on B-mode images in accordance with the disclosed subject matter.

FIG. 3 provides a graph showing Young's modulus measured in the normal pancreas in accordance with the disclosed subject matter.

FIGS. 4A-41 provide images showing Young's modulus maps of post=surgical human pancreas in accordance with the disclosed subject matter.

FIG. 5 provides a graph showing Young's modulus estimation using the disclosed HME technique in 35 post-surgical human specimens in accordance with the disclosed subject matter.

FIGS. 6A-6F provides photographic and H&E staining images showing pancreatic specimens with/without chemotherapy history in accordance with the disclosed subject matter.

FIGS. 7A-7L provide images showing different fibrosis stages using Mason's trichrome in accordance with the disclosed subject matter.

FIGS. 8A-8AA provide images showing human pancreatic specimens in accordance with the disclosed subject matter.

FIGS. 9A-9D provide graphs showing measured Young's modulus in accordance with the disclosed subject matter.

FIG. 10A-10D provide unoverlaid/overlaid images of reconstructed Young's modulus map in accordance with the disclosed subject matter.

FIGS. 11A-11C provide a B-mode image, a displacement image, and Young's modulus map in accordance with the disclosed subject matter.

FIG. 12 provides images showing Young's modulus map of a pancreatic tumor in accordance with the disclosed subject matter.

FIGS. 13A-13B provide a B-mode image of the tumor and a graph showing the absolute peak-to-peak displacement and Young's modulus in accordance with the disclosed subject matter.

FIGS. 14A-14B provide a B-mode image of the tumor and a graph showing the absolute peak-to-peak displacement and Young's modulus in accordance with the disclosed subject matter.

FIGS. 15A-15C provide images showing an overlay of Young's modulus map of the liver specimen before and after ultrasound application in accordance with the disclosed subject matter.

FIGS. 16A-16B provide an image of displacement map after ultrasound application and a graph showing Young's modulus profiles in accordance with the disclosed subject matter.

FIGS. 17A-17B provide images showing Young's modulus map on the original B-mode in accordance with the disclosed subject matter.

FIGS. 18A-18D provide images showing post-surgical pancreas specimen and Young's modulus map in accordance with the disclosed subject matter.

FIG. 19 provides a graph showing Young's modulus after HME application on 19 specimens in accordance with the disclosed subject matter.

It is to be understood that both the foregoing general description and the following detailed description are exemplary and are intended to provide further explanation of the disclosed subject matter.

### DETAILED DESCRIPTION

The disclosed subject matter provides techniques for harmonic motion elastography (HME). The disclosed subject matter provides systems and methods for measuring a mechanical property of a target tissue using the HME technique.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

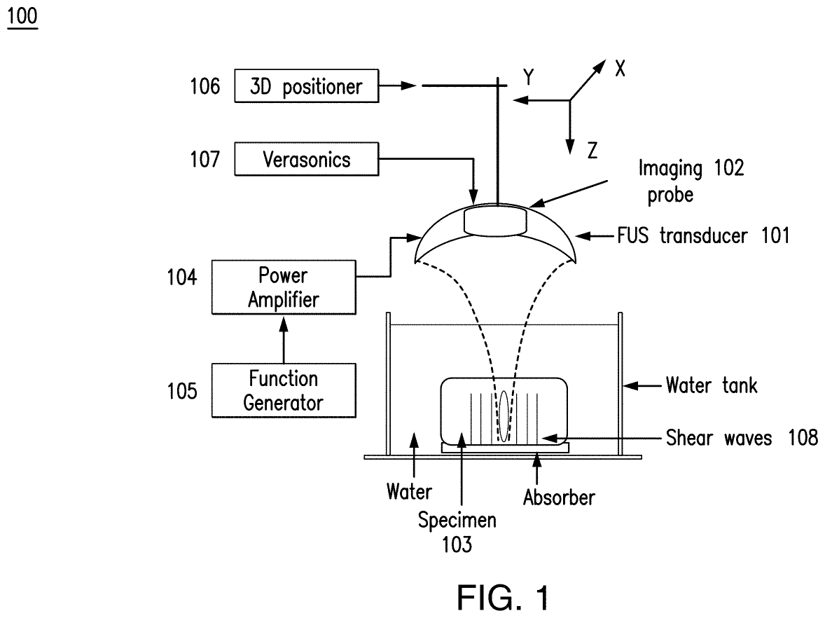

In certain embodiments, the disclosed subject matter provides a system for opening target tissue. As shown in FIG. 1, an example system 100 can include a focused ultrasound (FUS) transducer 101, an imaging transducer 102, and a processor. In non-limiting embodiments, the imaging transducer can be located in the middle of the FUS transducer. In some embodiments, the processor can be coupled with the FUS transducer and/or the imaging transducer. For example, the processor can be coupled to the probes directly (e.g., wire connection or installation into the probes) or indirectly (e.g., wireless connection).

In certain embodiments, the FUS transducer can be used to stimulate the target tissue by generating an acoustic radiation force. For example, the FUS transducer can generate and apply a push to the target tissue 103. The push can be any mechanical movement, deformation and/or momentum that the tissue undergoes as a result of FUS application. In non-limiting embodiments, the push can be an oscillatory radiation force that can generate deformation (e.g., the oscillatory motion) from the target tissue. For example, the oscillatory radiation force can be described by

$$F = \frac{2 α I}{C} \qquad ( 1 )$$

F is the generated radiation force (N), α is the tissue absorption coefficient (m⁻¹), I is the average acoustic intensity (W m⁻²), and c is the sound speed (m s⁻¹).

In non-limiting embodiments, the oscillatory motion can be harmonic displacement (e.g., oscillation around the initial location of the tissue prior to the FUS application). For example, the FUS transducer can generate the harmonic displacement on the target tissue without damaging the target tissue. The displacement can range from about 0.01 μm to about 300 μm, from about 0.01 μm to about 250 μm, from about 0.01 μm to about 200 μm, from about 0.01 μm to about 150 μm, from about 0.01 μm to about 100 μm, from about 0.01 μm to about 50 μm, from about 0.01 μm to about 40 μm, from about 0.01 μm to about 30 μm, from about 0.05 μm to about 25 μm, or from about 1 μm to about 25 μm.

In certain embodiments, the FUS transducer can be set with different combinations of ultrasound parameters for generating the push. The ultrasound parameters can include an acoustic intensity, a stimulation duration, duty cycle, pulse duration, and/or a center frequency. In non-limiting embodiments, the acoustic intensity can range from about 1 W/cm²— about 3000 W/cm², from about 1 W/cm²— about 2000 W/cm², from about 1 W/cm²-about 1500 W/cm², from about 1 W/cm²— about 1050 W/cm², from about 100 W/cm²-about 1050 W/cm², from about 200 W/cm²— about 1050 W/cm², or from about 500 W/cm²-about 1050 W/cm². In non-limiting embodiments, the stimulation duration can range from about 0.1 second to about 100 seconds, from about 1 second to about 100 seconds, from about 5 seconds to about 100 seconds, from about 10 seconds to about 100 seconds, from about 15 seconds to about 100 seconds, from about 20 seconds to about 100 seconds, from about 25 seconds to about 100 seconds, from about 30 seconds to about 100 seconds, from about 35 seconds to about 100 seconds, from about 40 seconds to about 100 seconds, from about 45 seconds to about 100 seconds, from about 50 seconds to about 100 seconds, from about 55 seconds to about 100 seconds, from about 60 seconds to about 100 seconds, from about 65 seconds to about 100 seconds, from about 70 seconds to about 100 seconds, from about 75 seconds to about 100 seconds, or from about 80 seconds to about 100 seconds. In non-limiting embodiments, the center frequency of the FUS stimulation probe can range from about 1 megahertz (MHz) to about 10 MHz, from about 1 MHz to about 9 MHz, from about 1 megahertz (MHz) to about 8 MHz, from about 1 megahertz (MHz) to about 7 MHz, from about 1 megahertz (MHz) to about 6 MHz, from about 1 megahertz (MHz) to about 5 MHz, from about 2 megahertz (MHz) to about 5 MHz, or from about 3 MHz to about 5 MHz. In some embodiments, the center frequency of the FUS stimulation probe can be about 4.5 MHz. In certain embodiments, the ultrasound parameters can be pre-programmed and/or adjusted depending on the target tissue or subject. For example, the FUS transducer can be a 93-element FUS transducer with about 4.5 MHz center frequency for stimulating soft tissues.

In certain embodiments, the FUS transducer is configured to move in a raster scanning manner.

In certain embodiments, the disclosed system can further include a power amplifier 104, a function generator 105, a 3D positioner 106, and a combined transducer 107. Positioner can be a system that moves the transducer in space in three dimensions. The combined transducer can be an ultrasound transducer to send and receive ultrasound signals or waves. The power amplifier 104 can amplify an amplification (e.g., 50-dB) of the signal generated by the function generator 105 before application onto the FUS transducer.

In certain embodiments, the disclosed system can include an imaging transducer. The imaging transducer can be used for obtaining ultrasound images and radio frequency (RF) signals of the target tissue. The imaging transducer probe can also be used for locating target tissue, monitoring stimulation/modulation of the target tissue, and acquiring RF signals from the target tissue before/after the FUS stimulation. For example, the oscillatory motion generated by the FUS transducer can be estimated by the channel data acquired by the imaging transducer. In non-limiting embodiments, the imaging transducer can be linear, curved, phased, 1D, or 2D array with a number of elements varying from 32 to 1024 elements. In some embodiments, the imaging transducer can be a 64-element phased-array imaging transducer or a 104-element diagnostic transducer.

In certain embodiments, the imaging probe can have at least one ultrasonic parameter. The ultrasound parameter can include a center frequency, bandwidth, element pitch, number of elements and/or frame rate. For example, the center frequency of the imaging transducer can range from about 4 megahertz (MHz) to about 16 MHz, from about 4 MHz to about 15 MHz, from about 4 MHz to about 14 MHz, from about 4 MHz to about 13 MHz, from about 4 MHz to about 12 MHz, from about 4 MHz to about 11 MHz, from about 4 MHz to about 10 MHz, from about 4 MHz to about 9 MHz, from about 4 MHz to about 8 MHz, from about 4 MHz to about 7 MHz, from about 4 MHz to about 6 MHz, or from about 4 MHz to about 5 MHz. In some embodiments, the center frequency of the imaging probe can be about 2.5 MHz or 7.8 MHz. In non-limiting embodiments, the imaging transducer can record a waveform generated by the FUS transducer. For example, the imaging transducer can record the waveform at about 100, about 300, about 500, about 1000, about 2000, about 3000, about 4000, about 5000 frames/second through the application of the acoustic force generated by the FUS transducer. In non-limiting embodiments,

In certain embodiments, the ultrasound parameters of the imaging transducer can be pre-programmed and/or adjusted depending on the target tissue or subject. For example, a 64-element imaging transducer with about 2.5 MHz center frequency can be used for phantom models. The phantom models can include tissue-mimicking organic (e.g., agar, gelatin) or inorganic (e.g., silicone) blocks. A 104-element imaging transducer with about 7.9 MHz center frequency can be used for animal or human subjects. In non-limiting embodiments, the imaging transducer can be configured to obtain radio frequency data in real-time.

In certain embodiments, the disclosed system can include a processor coupled to the FUS transducer and/or the imaging transducer. The processor can be configured to perform the instructions specified by software stored in a hard drive, a removable storage medium, or any other storage media. The software can include computer codes, which can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components described above. For example, and as embodied herein, the processor can be configured to control imaging and ultrasound stimulation. Additionally, or alternatively, the processor can be configured to control the output of the function generator and/or the transducer to provide the FUS to the subject. In some embodiments, the processor can be a Graphical Processing Unit (GPU) or be implemented in GPU.

In certain embodiments, the processor can be configured to reconstruct each RF data frame of the target tissue. For example, the RF channel data can be multiplied by a sparse matrix in which the data are sampled (e.g., at 80 MHz for 64-elements phased array or at 125 MHz for 104-element), and the product can be multiplied by another sparse matrix for scan conversion. In non-limiting embodiments, the processor can be configured to perform beamforming of the RF signals. In some embodiments, the processor can filter the beamformed RF data using a filter at the predetermined frequency. For example, the predetermined frequency can include fundamental and harmonic frequencies of the FUS stimulation transducer.

In non-limiting embodiments, the processor can calculate the displacement of the target tissue by performing 1D cross-correlation, which can include an estimate of the correlation between two signals acquired during different time points in the same region in the FUS stimulated tissue. For example, axial displacements at the focal point can be estimated by applying a 1D normalized cross-correlation on the reconstructed RF data. The cross-correlation estimation provides how much the tissue is displaced in comparison to a prior time.

In certain embodiments, the processor can be configured to generate a property map based on the RF signals. For example, the processor can generate Young's modulus map. In non-limiting embodiments, to generate the 2D Young's modulus map, the processor can extract shear wave 108 from the RF signals. As a complex field of shear waves can be generated due to constructive and destructive interaction of forward and reflected shear waves, a directional filter can be used to separate shear waves from such a complex field. The directional filter can separate waves that propagate in opposing or different directions. The directional filter can be designed in frequency space with the ability to choose the portion of the wave in a certain direction. In some embodiments, the standing wave can be reduced by applying the disclosed directional filter. Standing waves can be waves that are stationary in space due to large reflections. The standing waves can be combined with the incident waves and cancel out its propagation in space.

In certain embodiments, the disclosed processor can be configured to apply a time-of-flight algorithm to the beamformed RF signals to measure the time delay of the shear wave propagation by cross-correlating the filtered particle displacement profiles along the lateral direction. Then, two points separated (e.g., by eight for phantom model and six ultrasound wavelengths for clinical model) at the same depth can be used to calculate the traveling time of the shear wave to travel between these two points. Then, based on the estimated time delay between these points at a known distance, the shear wave speed can be measured. The measured shear wave can be assigned for the center pixel of the grid. In non-limiting embodiments, the shear wave map can be generated for each measurement, and Young's modulus can be reconstructed based on that.

In certain embodiments, the disclosed processor can distinguish a lesional part and a non-lesional part from the target tissue. For example, the disclosed processor can distinguish a lesional part and a non-lesional part based on the difference of mechanical properties. For example, tumor part, perilesional part, and non-cancerous par can be identified based on the significant differences in their measured Young's modulus or stiffness. The tumor part can demonstrate a relatively high Young's modulus value than the non-cancerous part. In non-limiting embodiments, the perilesional part can be stiffer than the non-cancerous part and softer than the tumor part. In some embodiments, the disclosed processor can identify a boundary between the lesional part and the non-lesional part.

In non-limiting embodiments, the mechanical properties can include elasticity, stiffness, viscosity, poroelasticity, or combinations thereof.

In certain embodiments, the target tissue can be any tissues. For example, the target tissue can be a nerve, a brain, a heart, muscle, tendons, ligaments, skin, vessels, tumor, cancer, or a combination thereof.

In certain embodiments, the disclosed subject matter provides a method for measuring a mechanical property of target tissue. An example method can include modulating the target tissue by inducing a push with the disclosed focused ultrasound (FUS) ultrasound, obtaining radio frequency (RF) signals from the target tissue using the disclosed imaging transducer, and estimating a mechanical property based on the RF signals. In certain embodiments, the ultrasound parameters of the FUS transducer and the imaging transducer can be pre-programmed and/or adjusted depending on the target tissue or subject. For example, the FUS transducer can be a 93-element FUS transducer with about 4.5 MHz center frequency for stimulating soft tissues. A 64-element imaging transducer with about 2.5 MHz center frequency can be used for phantom models. A 104-element imaging transducer with about 7.9 MHz center frequency can be used for animal or human subjects. In non-limiting embodiments, the focused ultrasound can be moved in a raster scanning manner. In certain embodiments, the RF signals can be obtained through a single push. For example, after one tissue region moving mechanically, the RF signals can be acquired before, during, and after.

In certain embodiments, the method can further include adjusting the frequency of the push-based on the target tissue. The frequency can range from 0.1 Hz to 100 kHz.

In certain embodiments, the method can further include conducting beamforming on the RF signals. The RF signals can be reconstructed based on the channel data using beamforming techniques such as the delay and sum method. In non-limiting embodiments, the method can also include filtering the beamformed RF data using a filter at the predetermined frequency.

In certain embodiments, the method can further include estimating RF displacement of the target tissue through a 1D cross-correlation. For example, axial displacements can be estimated by applying a 1D normalized cross-correlation on the reconstructed RF data for identifying how much the tissue is displaced in comparison to a prior time.

In certain embodiments, the method can further include extracting shear wave from the estimated RF displacement and estimating shear wave speed. For example, a directional filter can be used to separate shear waves from the RF signals. The shear wave can be estimated by the displacement and its traveling mechanism in space from the source. The displacement map can be tracked over time to determine the shear waves that emanate from the focus in the tissue. The directional filter can be designed in frequency space with the ability to choose the portion of the wave in a certain direction. In some embodiments, the method can further include applying the directional filter for reducing standing waves.

In certain embodiments, the method can further include calculating Young's modulus using the estimated shear wave speed and generating Young's modulus map. For example, a time-of-flight algorithm can be applied to the beamformed RF signals to measure the time delay of the shear wave propagation by cross-correlating the filtered particle displacement profiles along the lateral direction. The time of flight can indicate the assessment of the propagation characteristics such as the distance traveled by the shear wave, its velocity, and/or acceleration. Based on the estimated time delay between these points at a known distance, the shear wave speed can be measured. In non-limiting embodiments, the shear wave map can be generated for each measurement, and Young's modulus can be reconstructed based on the measured shear wave speed.

In certain embodiments, the method can further include identifying a boundary between a lesion area and a non-lesion area of the target tissue. In non-limiting embodiments, the lesional part and the non-lesional part can be identified based on the difference in mechanical properties. For example, tumor part, perilesional part, and non-cancerous par can be identified based on the differences in their measured Young's modulus or stiffness.

### EXAMPLES

### Example 1: Harmonic Motion Elastography (HME) and its Application in In-Vivo Young's Modulus Visualization of Pancreatic Ductal Adenocarcinomas in Transgenic Mice and Human Specimens

Harmonic Motion Elastography (HME): the confocal configuration of this system is illustrated in FIG. 1. This system includes a 93-element HIFU transducer, FUS, (fc=4.5 MHz, and D=70 mm, Sonic Concepts Inc., Bothell Wash., USA). For the imaging part, either a 64-element phased-array imaging probe (fc=2.5 MHz, P4-2, ATL/Philips, Bothell, Wash., USA) using for human pancreatic specimen or a 104-element diagnostic transducer (fc=7.8 MHz, P12-5, ATL/Philips, Bothell, Wash., USA) using for mice model is used. Either of these imaging transducers was confocally aligned with the HIFU transducer. FUS transducer is derived by an AM sinusoidal signal generated by a dual-channel arbitrary waveform generator (AT33522A, Agilent Technologies Inc. Santa Clara, Calif., USA) through a 50 dB power amplifier (325LA, E&I, Rochester, N.Y., USA). The total acoustic power output of the FUS transducer is in the range of 6.4-8.6 W from radiation force balance measurements.

To reduce the breathing motion artifact in vivo mice model while recording the data, a respiration gating system (Biopac System, Santa Barbara, Calif., USA) is used. The pressure sensor of this unit is connected to MP150 Data Acquisition System. The output of this is used to trigger the vantage system to synchronize the sonication and data acquisition.

The generated oscillatory motion by HIFU transducer is recorded by imaging transducer connected to an ultrasound imaging research system (Vantage, Verasonics, Bothell, Wash., USA) in the form of Radio Frequency (RF) channel data with a sampling frequency of 1000 frames per second. For imaging reconstruction, the RF channel data is multiplied by a sparse matrix in which the data are sampled at either 80 MHz for 64-elements phased array or 125 MHz for 104-element, and the product is multiplied by another sparse matrix for scan conversion. A graphical Processing Unit (GPU) is used for this beamforming process.

The 1-D normal cross-correlation is applied on beamformed RF data, and then the 2D directional filter and time of flight algorithm are used to measure the shear wave speed and Young's modulus estimation. The window size using for the mice model is six pixels and for post-surgical specimens is eight pixels for In vivo Young's modulus assessment on transgenic mice

A total of 44 mice (aged 8-20 weeks) were used. Within less than one hour, 1-2% isoflurane in oxygen was used to anesthetize the animal and left on a heating path in a supine position. A container with a transparent acoustical window and full of degassed water was placed on the mice's abdomen, and the gap was filled with ultrasound gel.

One push was applied in the center of the pancreas to generate displacement and Young's modulus 2D map. To locate the pancreas and other organs in mice, a higher frequency transducer (L22-14v, fc=18.5 MHz, Verasonics) imaged the pancreases before using HME.

Wild type, Pancreatic, and PDA mice: Among the 44 mice used, the control group included 15 BalbC mice, wild type mice, and the rest that were genetically engineered, used as a testing group. The testing group also contained two main subgroups: chronic pancreatitis and PDA ones.

There were a total of 25 chronic pancreatitides or KC (K-rasLSL.G12D/+; PdxCretg/+) mice. Generally, these premalignant pancreatic tumors are the potential to be transformed into Pancreatic Ductal Adenocarcinoma (PDA) in mice with more than one-year-old. Pancreatitis or inflammation is a common sign of the pre-development of a pancreatic tumor. To assess the mechanical alteration happening during this transitional stage, the aforementioned tumor-free KC mice, 8-20 weeks old, were injected with 250 mg kg −1 of cerulein for 5 days to intensify chronic pancreatitis in them. Then, one week after injection, they were scanned. After scanning, the mice went through the euthanizing and necropsying process. Then, the pancreas was harvested and prepared for H&E staining. The blinded expert observed the entire pancreas slides under the microscope with X 4 and X 10 magnification. The fibrosis percentage of each slide was calculated based on the overall pancreatic slide surface. Finally, these resulted fibrosis percentages were used to categorize the mice in two groups: less than 50% fibrosis and over 50% fibrosis.

In addition to KC mice, there were five cases of KPC (K-rasLSL.G12D/+; p53LSL.R172H/+; PdxCretg/+) mice. This transgenic mouse model has the ability to develop the PDA in less than 6 months. The tumor's diameter is usually between 3-5 mm. The unique characteristic of such mice is their high resemblance to human PDA from both physiological and molecular perspectives.

Young's modulus assessment on human pancreatic specimen: The post-surgical PDA specimens were collected to measure the young's modulus of these solid carcinomas using the HME method. The 35 specimens were immersed in PBS degassed water tank. An absorber was placed underneath the specimen to reduce the reflected echoes.

Despite all the experiments using a single push so far, the PDA human specimens were scanned in raster mode to cover the whole specimen. The 3D positioner was moved almost 3 mm for each scan in axial and lateral directions, and the overall Young's modulus 2D map was reconstructed based on the Young's modulus determined at each scan.

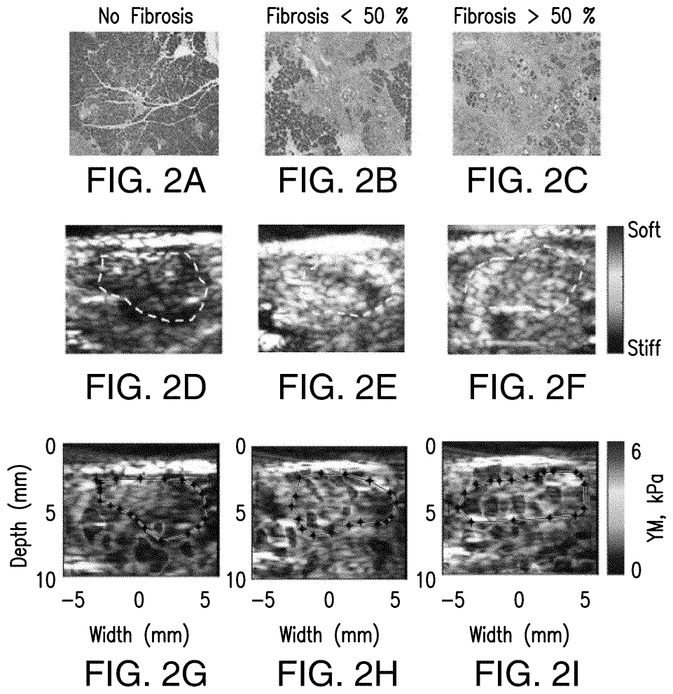

In vivo Young's modulus assessment of PDA solid tumors in transgenic mice: FIG. 2 demonstrates the result of applying the HME method in vivo mice model with different level of fibrosis including the mice with normal pancreas, the ones with less than 50% fibrosis, the ones with more than 50% and finally the ones with full-fledged pancreatic tumors. The hematoxylin and eosin staining (H & E) images related to each group also was added to FIG. 2.

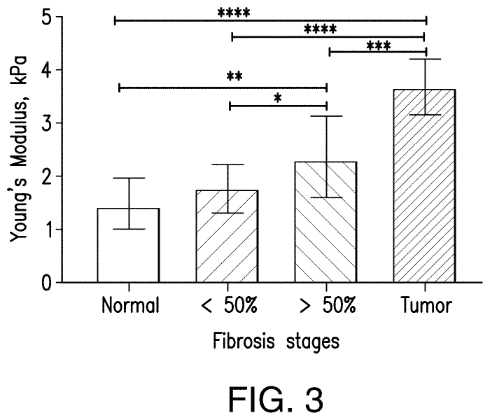

HMI was capable of differentiating between fibrosis levels in mice based on the local displacement measurement. It is shown that the HME can estimate Young's modulus at each fibrosis level, and the results corroborate well with what HMI predicted. To create the 2D peak-to-peak displacement map illustrated in FIG. 2, the raster scanning, using multiple pushes, is used while in one single push was applied to generate these 2D Young's modulus maps using the HME techniques. FIG. 2A shows a 2D HMI displacement map of the pancreas with no fibrosis specified with the dashed region 201. The estimated median displacement is 16.1 um. FIG. 2B shows a 2D HMI displacement map of the pancreas with less than 50% fibrosis specified with the dashed region 202. The estimated median displacement is 6.8 um. FIG. 2C shows a 2D HMI displacement map of the pancreas with more than 50% fibrosis specified with the dashed region 203. The estimated median displacement is 5.8 um. FIG. 2D shows 2D Young's modulus map of the pancreas with no fibrosis. The estimated median Young's modulus of the specified area 204 is 1.2 KPa. FIG. 2E shows 2D Young's modulus map of the pancreas with less than 50% fibrosis specified with the dashed region. The estimated median Young's modulus of the specified area 205 is 2.1 KPa. FIG. 2F shows 2D Young's modulus map of the pancreas with more than 50% fibrosis specified with the dashed region 206. The estimated median Young's modulus of the specified area is 3.5 KPa. FIG. 3 shows the HME application summary on 44 mice at different levels of fibrosis, as mentioned before.

Young's modulus estimation of PDA in post-surgical human specimens, including its tumor, perilesional and non-cancerous part: This new shear-wave based elastography method (HME) is applied on some post-surgical human specimens with pancreatic tumors. These specimens are categorized into three groups; the first group contains 19 specimens without having any treatment experiences like chemotherapy before surgery. The second one is the group of 14 specimens who experienced the treatment procedure like chemotherapy before surgery, and the third one includes 2 specimens classified as Intraductal papillary mucinous neoplasm (IPMN), which can be transformed to malignancy.

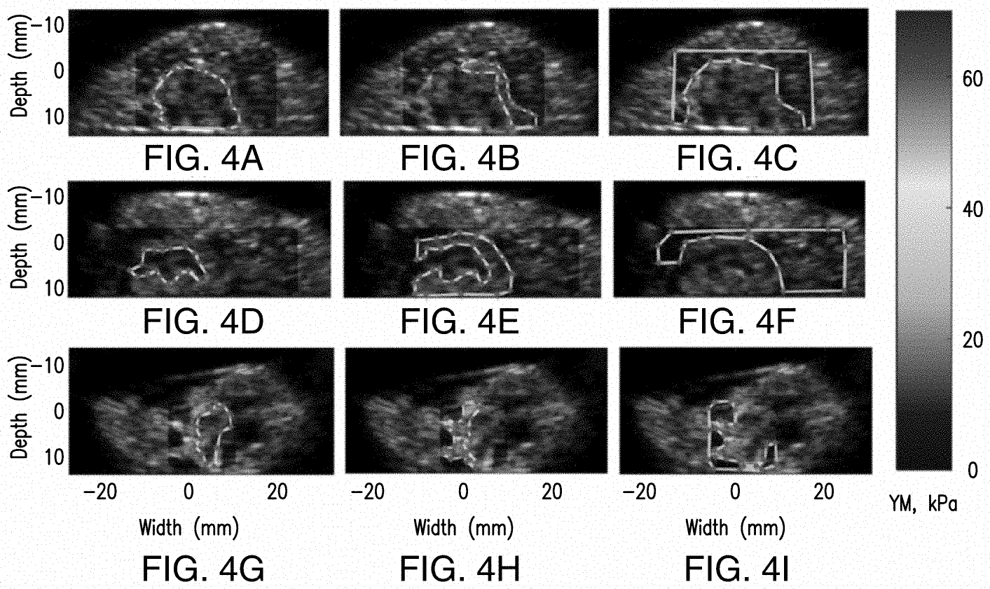

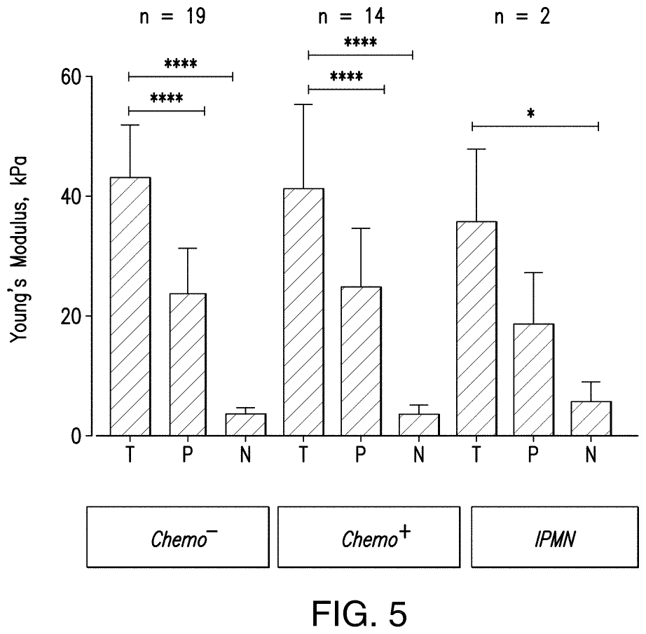

Furthermore, it is shown that each pancreatic specimen, no matter from which group, can be divided into three parts: tumor part, perilesional part, and non-cancerous one because of the significant difference in their measured Young's modulus or stiffness. The tumor part demonstrates the largest Young's modulus value while the non-cancerous part has the lowest one, and the perilesional part is always stiffer than the non-cancerous part and softer than the tumor part. FIG. 4 illustrates the HME method application on post-surgical specimens. In FIG. 4, the first column represents the tumor region of the specimens, and the second and third ones show the perilesional and non-cancerous regions, respectively. In addition, the measured median YM value related to each part based on the specified region is disclosed. The same technique was applied to all post-surgical human pancreatic specimens, and the result is summarized in FIG. 5. This figure shows there is a significant difference based on YM estimation between different parts of specimens in each group. However, there are no significant differences between the three groups. FIG. 4A shows the tumor part of the pancreatic specimen with no chemotherapy history. The estimated median YM value of the specified region 401 is 47.1 KPa. FIG. 4B shows the perilesional part of the pancreatic specimen with no chemotherapy history. The estimated median YM value of the specified region 402 is 19.9 KPa. FIG. 4C shows the non-cancerous part of the pancreatic specimen with no chemotherapy history. The estimated median YM value of the specified region 403 is 4.1 KPa. FIG. 4D shows the tumor part of the pancreatic specimen with chemotherapy history. The estimated median YM value of the specified region 404 is 52.4 KPa. FIG. 4E shows the perilesional part of the pancreatic specimen with chemotherapy history. The estimated median YM value of the specified region 405 is 30.3 KPa. FIG. 4F shows the non-cancerous part of the pancreatic specimen with chemotherapy history. The estimated median YM value of the specified region 406 is 4.1 KPa. FIG. 4G shows a tumor part of the IPMN specimen. The estimated median YM value for the specified region 407 is 45.7 KPa. FIG. 4G shows a perilesional of IPMN specimen. The estimated median YM value for the specified region 408 is 27.1 KPa. FIG. 41 shows a non-cancerous part of the IPMN specimen. The estimated median YM value for the specified region 409 is 5.6 KPa.

Microscopic analysis using the H&E method: In order to identify the correlation between structural changes in the cellular level and the local stiffness as reported by the 2D YM map, right after scanning, the specimens go through the pathological analysis, the same as what has been done in mice model.

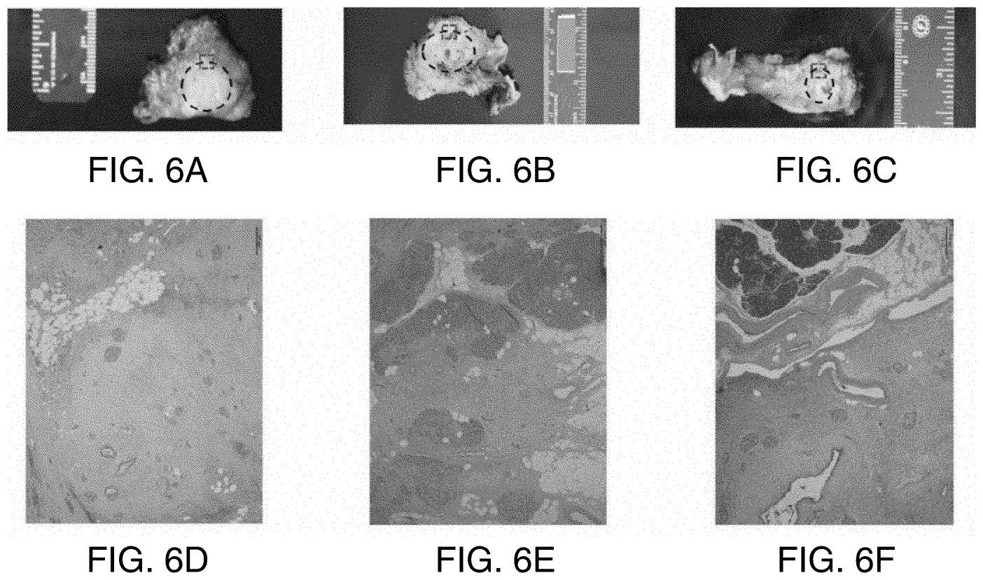

FIG. 6 shows the cutting plane pictures of the specimens with their H&E images. Black dashed lines 601, 602, 603 in this figure illustrate the tumor part and the purple squares represent the approximate region that H&E slides come from. FIGS. 6A-6C shows cutting plane pictures of the pancreatic specimen with no chemotherapy history (FIG. 6A), with chemotherapy (FIG. 6B), and IPMN specimen (FIG. 6C). FIGS. 6D-6F show H&E staining of the pancreatic specimen with no chemotherapy history (FIG. 6D), with chemotherapy (FIG. 6E), and IPMN specimen (FIG. 6F).

The H&E images contain both the tumor region and the perilesional one. These specimens are the ones that their 2D YM maps have been shown in FIG. 4. Moreover, these cutting plane pictures are the most similar plane using to create 2D YM maps in FIG. 4.

The development of extremely fibrotic stroma in PDA pancreatic tumors is due to the dense and cross-linked extracellular matrix (ECM) that occurs as the disease progress. Certain liver cirrhosis and breast models demonstrate the direct correlation between fibrosis and the risk of cancer occurrence. Fibrosis promotes the stiffness elevation in the stroma, and this newly developed rigid stroma promotes tensional homeostasis that culminates in a high level of cell contractility. For example, in breast cancer, ECM was stiffening and collagen crosslinking triggers the formation of integrin containing focal adhesions in the cell membrane. This can result in intracellular signaling accompany by an extracellular signal-regulated kinase that leads to ROCK-generated contractility and malignancy. In addition, matrix stiffness can play a significant role in promoting resistance against chemotherapeutics. Thus stiffness estimation during tumor progression not only can assist in prescribing the effective treatment based on the tumor stiffness but also is able to provide a solid evaluation of treatment efficiency in post-treated tumors based on the stiffness change.

The disclosed HME method was used to assess its performance in classifying the pancreatic tumors in transgenic mice with various fibrosis levels. The results showed that there was a solid correlation between the reported Young's modulus value using HME and the fibrosis level based on the pathology report. The higher Young's modulus, in other words, stiffer tumor, belonged to the tumor with higher fibrotic status. Another reference to assess the HME method was the 2D peak-to-peak maps generated based on the same acquired data in these mice models. The 2D Young's modulus maps also corroborated well with 2D peak-to-peak maps. FIG. 3 showed a summary of this mice model. Although HME was not able to differentiate between the normal group and the group with less than 50% fibrosis, there is a significant difference in estimated YM in normal and other groups and between groups with various fibrosis percentage.

Despite using a single push for mice model to generate the 2D Young's modulus maps, raster scanning was applied for the post-surgical human model. Definitely, this way of scanning enables the HME to report the mechanical properties of tumors in a more accurate and precise way comparing to other shear wave-based methods using a single push. This method, HME, helps in the detection of three different regions in each specimen, as it is shown in FIG. 4. In other words, this high-resolution scanning method not only has the ability to detect and estimate Young's modulus value of the tumor and its non-cancerous (background) part, but also the transient area (perilesional region) between these regions are conspicuous in reconstructed 2D YM maps. FIG. 5 presents the stiffness estimation of such regions. The estimated YM for the tumor part (T) and the noncancerous region (N) is more than 30 kPa and less than 10 kPa, respectively. However, for the perilesional region (P), the estimated YM is somewhere between 15 to 30 kPa approximately. This unique characteristic of this method can provide a more reliable method for the surgeon to delineate the tumor boundaries while doing surgery and can decrease the surgeon's reliance on pathology laboratory during surgery. In addition, it can help to decrease the chance of leaving the tumor part in tissue after surgery. This can help to reduce the recurrence of such aggressive pancreatic tumors.

In spite of the aforementioned advantages, the scanning time for these multiple push-based methods took longer than a single one. In addition, there was a limited time between receiving the specimen, doing the scan, and bringing back to the pathology department. Due to this time limitation for scanning, it was not possible to scan the whole lesion or using 3D scanning. Thus, the preference was to scan the tumor and its surrounded regions. This is the reason that the reconstructed 2D maps of each specimen (FIG. 4) does not cover the whole specimen, but it is enough to show the stiffness changes from non-cancerous region to perilesional and tumor part. There are no criteria observed regarding the perilesional area's stiffness value. The disclosed subject matter was used to measure Young's modulus value for the perilesional regions.

In addition, inhomogeneity or discrete patterns can be observed in YM maps, FIGS. 4 and 5, in both mice and specimens, this pattern is more pronounced in PDA tumors in mice rather than in specimens. The dissection of the organ can change its mechanical properties; thus, the specimen mostly contains the solid part of the tumor, and the fluid part disappears during the dissection.

Furthermore, the treatment methods using for a patient with chemotherapeutic history were not effective in softening the tumor. FIG. 5 shows that there is no significant difference in estimated stiffness in specimens with chemotherapy history and the ones without chemotherapy history. In other words, the treatment method did not assist in softening the PDA tumors.

The disclosed subject matter can be used as a prognosis imaging in patients with PDA tumors to assess the stiffness of the tumor and evaluate the efficiency of the treatment methods. The disclosed subject matter can also pave the route to measure the stress and pressure inside the tumors.

The performance of the HME method was evaluated on PDA tumors in transgenic mice and later on, post-surgical PDA tumors. The results show that the HME is capable of differentiating between PDA tumors with various fibrosis levels. In addition, in post-surgical specimens, three different religions classified as the tumor, perilesional part, and non-cancerous ones are recognized using HME methods.

### Example 2: Noninvasive Young's Modulus Visualization of Fibrosis Progression and Delineation of Pancreatic Ductal Adenocarcinoma (PDA) Tumors Using Harmonic Motion Elastography (HME) In Vivo

Harmonic Motion Elastography (HME): The HME system includes a 93-element FUS (Focused Ultrasound) transducer (fc=4.5 MHz, and D=70 mm, Sonic Concepts Inc., Bothell Wash., USA). For the imaging component, we used a 64-element phased-array imaging probe (fc=2.5 MHz, P4-2, ATL/Philips, Bothell, Wash., USA) to evaluate human pancreatic specimens and a 104-element diagnostic transducer (fc=7.8 MHz, P12-5, ATL/Philips, Bothell, Wash., USA) for the murine model. Each imaging transducer was confocally aligned with the HIFU transducer. The FUS transducer is derived by an AM sinusoidal signal generated by a dual-channel arbitrary waveform generator (AT33522A, Agilent Technologies Inc. Santa Clara, Calif., USA) through a 50 dB power amplifier (325LA, E&I, Rochester, N.Y., USA). In order to reduce the breathing motion artifact recorded during the in vivo mouse model, a respiration gating system (Biopac System, Santa Barbara, Calif., USA) is used. The pressure sensor of this unit is connected to the MP150 Data Acquisition System. Its output is used to trigger the ultrasound Verasonic system (Vantage, Verasonics, Bothell, Wash., USA) to synchronize sonication and data acquisition.

In vivo Young's modulus assessment of transgenic mice pancreata: For 45 in vivo mice pancreatic cases, within less than one hour, 1-2% isoflurane in oxygen was used to anesthetize the animal that was placed on a heating path in a supine position.

Mouse model characterization (chronic pancreatitis and PDA models): Among the 45 Young's modulus (YM) measurements on mice used, the control group included 15 YM measurements coming from wild-type BalbC mice. Another part of this experimental group is mice with chronic pancreatitis. There were a total of 25 YM measurements in mice with chronic pancreatitis or KC (K-rasLSL.G12D/+; PdxCretg/+) mice. These mice can develop pancreatic adenocarcinoma when they are more than one-year-old. These types of mice are susceptible to spontaneous inflammation with mild fibrosis, and at an advanced age, this inflammation can be transformed into PDA.

Histological analysis: Immediately after resection, the pancreatic specimens were immersed in cold 4% formaldehyde solution in phosphate-buffered saline (Affymetrix) at 4° C. Then, they were embedded in paraffin and cross-sectioned in 4 μm-thick sections. After deparaffinized, rehydrated and processed for routine staining with hematoxylin and eosin using the ST Infinity H&E staining kit (Leica).

Picrosirius Red (PSR): Picrosirius red (PSR) staining was used for fibrosis assessment because this method is capable of visualizing and quantifying the amount of collagen in tissue and report it as PSR density. As opposed to more traditional stains such as trichrome, Picrosirius red has selectivity that enables this method for both staining and quantification of collagen.

To perform Picrosirius red staining, 0.1% Sirius Red solution dissolved in aqueous saturated picric acid was used in this staining protocol. First, the slides were incubated in this solution then washed with 100% ethanol. They were dehydrated and mounted with Permount. After preparation, the freshly PSR stained slides were examined under polarizing light microscopy (Olympus BX41 TF) at a magnification of 4 X. For all images, the halogen lamp intensity was set constant and an exposure time within each image type was selected and kept constant to help to optimize the signal-to-noise ratio. CellSense acquisition platform (Olympus) was used to capture the images digitally. To analyze the captured images, Image J software was employed.

Young's modulus assessment of surgical human pancreatic specimens: Human pancreatic ductal adenocarcinoma surgical resection specimens were examined to measure Young's modulus using the HME method. There were a total 32 freshly cut specimens with PDA tumors. Among them, 17 cases were resected through pancereatomy, either distal or the whole pancreas, and 15 cases were obtained due to the Whipple procedure. The patient's range age was between 44 to 95 years old (68.8±9.3 years old). These specimens were immersed in a degassed, PBS-filled tank. An absorber was placed underneath the specimen to reduce reflection echoes.

The human PDA resection specimens were scanned in raster mode to cover the whole specimen. In each scan, the 3D positioner was moved 3 mm in axial and lateral directions, and the overall Young's modulus 2D map was reconstructed based on the resulted Young's modulus at each scan. Due to the time limitation for returning the specimens to the pathology department, the data acquisition should not take more than 90 minutes.

During imaging of these freshly excised specimens, the imaging probe was perpendicular to the pancreatic duct to have a solid correlation with histology slicing. In order to understand better the correlation between the structural changes, especially the rate of fibrosis at the cellular level and the local stiffness as reported by the 2D YM map, right after scanning, sections of the pancreas corresponding to the HME plane were submitted for histological examination. The histological examinations were performed on post-surgical human specimens, including the tumor part, it's perilesional, and its normal surrounding tissue.

In vivo Young's modulus assessment of PDA solid tumors in transgenic mice: Genetically engineered mice, the Kras^{LSL.G12D/+}; p53^{LSL.R172H/+}; PdxCre^{tg/+} (KPC) models were used. This type of transgenic mouse model is a well-established and clinically-predictive in vivo model of PDA. In this pancreas-specific mouse model, the endogenous expression of point-mutant K-ras and p53 are responsible for cognate mutations, and both of them play a key role in developing PDA in pancreatic patients. In the beginning, there was no sign of a PDA tumor in the newborn KPC mouse models, and all of them were born with normal pancreata. However, gradually, over several months, they develop more severe pathology. Initially, they develop acinar-to-ductal metaplasia (ADM), then pancreatic intraepithelial neoplasia (PanIN) lesions, and finally, they progress to overt ductal adenocarcinoma. These fibrosis progression increases more fibrotic extracellular matrix composition along with elevation in the solid stress and interstitial fluid pressure (IFP) as can cause changes in phenotype. Thus, this genetically engineered mouse model seems a solid fit to evaluate the HME performance in detecting the PDA tumor at different progression stages by measuring their stiffness noninvasively.

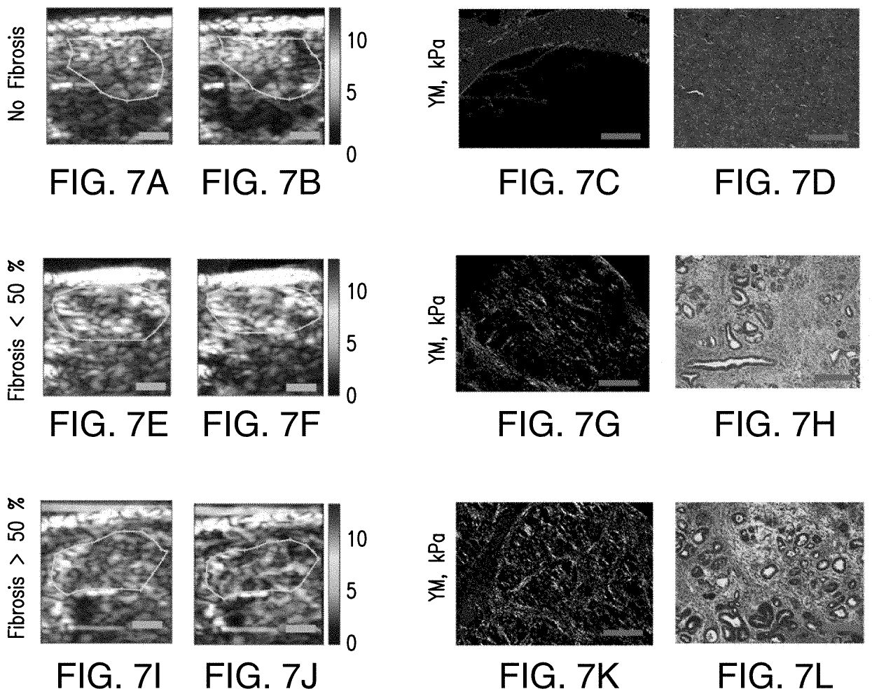

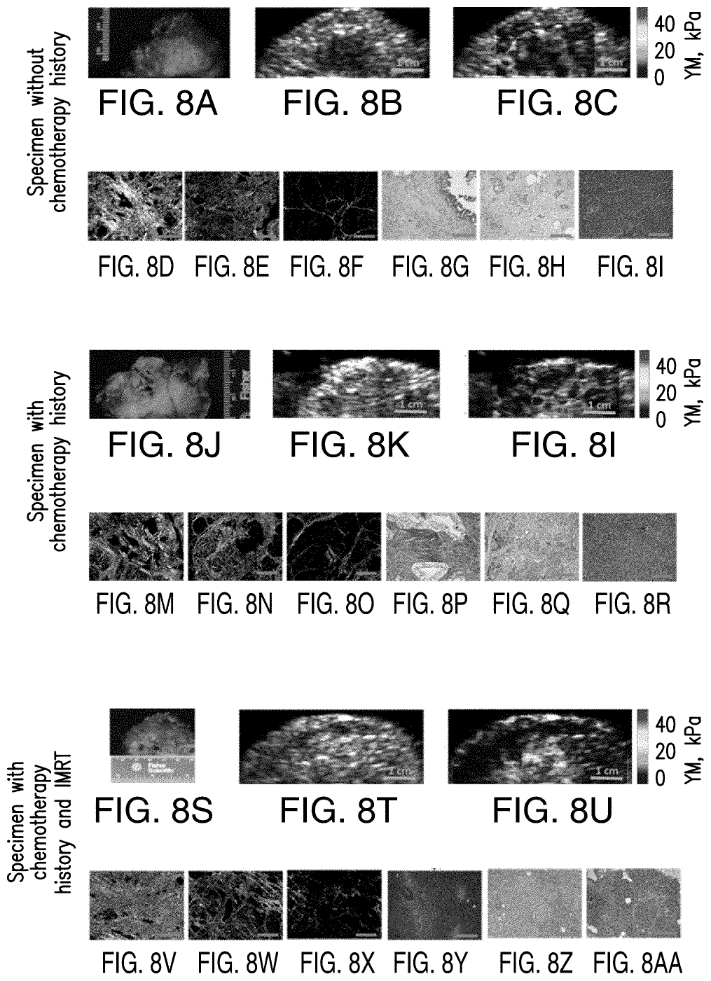

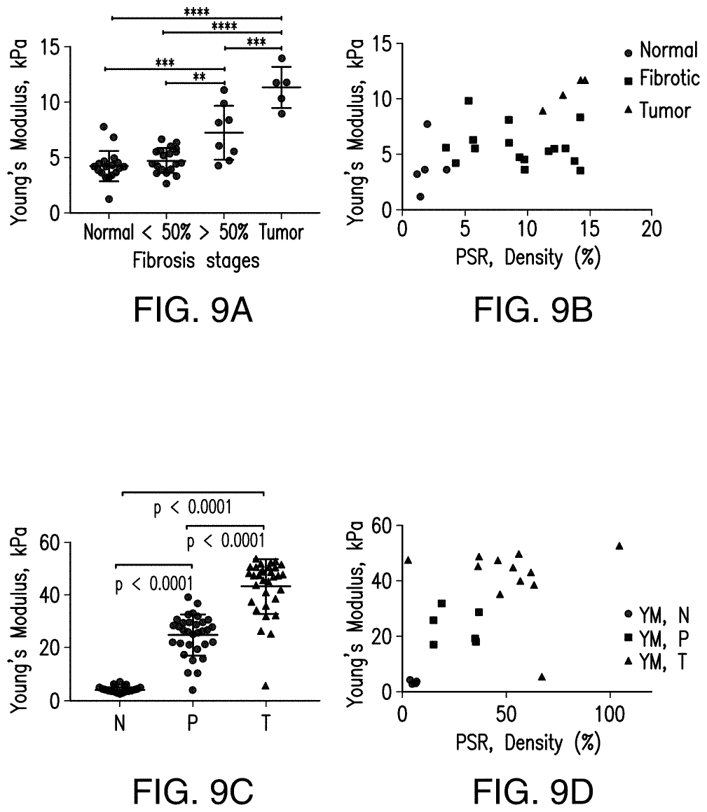

HME was applied to these in vivo mice with different levels of pancreatic fibrosis, including mice with normal pancreas, mice with <50% fibrosis, mice with >50% fibrosis, and finally mice with pancreatic tumors. FIG. 7 demonstrates the result of a mouse representing each group. The corresponding histological images accompanied by 2D Young's modulus map of each mouse is depicted. In the caption of this figure, the PSR density (collagen density) and Young's modulus related to each case is documented. FIGS. 7A-7L show different fibrosis stages using Mason's trichrome with 20× magnification, Picrosirius red staining method, and corresponding B-mode image and Young's modulus 2D maps overlaid on B-mode images. FIG. 7A shows a B-mode image of the pancreas, specified with contour 701, with no fibrosis. FIG. 7B shows a 2D Young's modulus map of the pancreas, specified with contour 702, with no fibrosis overlaid on a B-mode image. The estimated median of Young's modulus is (YM=3.6 kPa). FIG. 7C shows a picrosirius red slide of the pancreas with no fibrosis (PSR, density=4.2%). FIG. 7D shows Mason's trichrome slide of the pancreas with no fibrosis. FIG. 7E shows a B-mode image of the pancreas, specified with contour 703, with less than 50% fibrosis. FIG. 7F shows a 2D Young's modulus map of the pancreas, specified with contour 704, with less than 50% fibrosis overlaid on the B-mode image. The estimated median of Young's modulus is (YM=6.3 kPa). FIG. 8G shows a picrosirius red staining slide of the pancreas with less than 50% fibrosis (PSR, density=7.1%). Mason's trichrome slide of the pancreas with less than 50% fibrosis. FIG. 7H shows Mason's trichrome slide of the pancreas with less than 50% fibrosis. FIG. 7I shows a B-mode image of the pancreas, specified with contour 705, with more than 50% fibrosis. FIG. 7J shows a 2D Young's modulus map of the pancreas, specified with contour 706, with more than 50% fibrosis overlaid on B-mode image. The estimated median Young's modulus is (YM=11 kPa) specified with cyan contour. FIG. 7K shows a picrosirius red slide of pancreas with more than 50% fibrosis. The Picrosirius density is (PSR, density=12.1%). FIG. 7L shows a Mason's trichrome slide of pancreas with more than 50% fibrosis. In cases with no fibrosis, the acinar cells are the dominant part of the pancreatic tissue, with a flat epithelium and columnar cells, depicted in FIG. 7 (d). (YM=3.6 kPa, PSR=4.2%). For samples with fibrosis under 50%, the proportion of acinar cells is decreased, and the amount of fibrosis is increased. The alteration of supranuclear mucin into the papillary structure is apparent in FIG. 7 (h). (YM=6.3 kPa, PSR=7.1%). In the case of >50%, fibrosis is dominant, and fewer acinar cells are visible. In the corresponding Masson's trichrome, the abnormalities are more visible, and the polarity of the papillary structure is lost, and crowding occurs, FIG. 7 (l). (YM=11 kPa, PSR=12.1%), which is higher than the previous less fibrotic cases. FIG. 9 (a, b) and Table 1 summarize the HME findings at different levels of pancreatic fibrosis, as previously mentioned. As it was shown in FIG. 9 (b), we considered all the cases with <50% fibrosis and >50% fibrosis together as fibrotic cases, as shown in table 1.

TABLE 1
Summary of applying HME and Picrosirius
red method on transgenic mice

| Pancreta | | Young's modulus | PSR |
|---|---|---|---|
| type | Number | (HME), kPa | density (%) |
| Normal | 5 | 4.2 ± 1.3 | 2.0 ± 0.8 |
| Fibrotic | 17 | 5.5 ± 2.0 | 9.8 ± 3.4 |
| PDA | 4 | 11.3 ± 1.7 | 13.2 ± 1.2 |

Young's modulus assessment of PDA solid tumors in human surgical specimen: Based on the aforementioned animal findings of HME, the translational capability was assessed on surgical human pancreatic cancer specimens from PDA patients undergoing resection. Prior to imaging freshly resected specimens by HME, margins were assessed by the frozen section. During HME imaging of each specimen, first, several B-mode images were obtained continuously through the specimen in accordance with standard pathology procedures such that an approximate co-registration of imaging and histopathology can be performed. The acquired specimens were categorized into two groups. The first group contained 18 specimens with no prior treatment. The second group consisted of 14 specimens exposed to chemotherapy like gemcitabine/Abraxane (GA), gemcitabine/Taxotere/Xeloda (GTX), FOLFIRINOX (FOL: Leucovorin Calcium (Folinic Acid), F: Fluorouracil, IRIN: Irinotecan Hydrochloride, OX: Oxaliplatin), with or without radiotherapy, table 2.

TABLE 2
Patient Population and characteristics

| case# | Gender | Age | Tumor Site | Grade | T Stage | N Stage | Lymph Nodes Positive (P)/total | Margins | Type of Surgery | Neoajuvant Therapy |
|---|---|---|---|---|---|---|---|---|---|---|
| HMI-001 | F | 68 | Body/ Tail | Grade 2-3 | PT3 | pN1 | P = 12, T = 27 | Negative | Distal pancreatectomy | No prior therapy |
| HMI-003 | F | 67 | Body | Grade 1-2 | PT3 | pN1 | P = 1, T = 11 | Negative | Distal pancreatectomy | No prior therapy |
| HMI-005 | M | 67 | Body | Grade 2 | ypT1 | ypN0 | P = 0, T = 27 | Negative | Distal pancreatectomy | 4 months GTX + SBRT |
| HMI-007 | M | 67 | Body | Grade 2 | pT3 | pN0 | P = 0, T = 13 | Negative | Distal pancreatectomy | No prior therapy |
| HMI-009 | F | 62 | Tail | Grade 2 | pT3 | pN1 | P = 4, T = 16 | Negative | Distal pancreatectomy | No prior therapy |
| HMI-013 | F | 95 | Head | Grade 2-3 | pT3 | pN0 | P = 0, T = 21 | Negative | Total pancreatectomy | No prior therapy |
| HMI-018 | M | 70 | Head | Grade 2 | ypT3 | ypN1 | P = 1, T = 14 | Equivocal | Whipple | 4 months GTX + SBRT |
| HMI-020 | F | 73 | Body | Grade 3 | ypT3 | ypN1 | P = 3, T = 17 | Positive | Distal pancreatectomy | 2 months of gemsitabine-based |
| | | | | | | | | | | chemo |
| HMI-021 | M | 73 | Head | Grade 3 | ypT3 | ypN1 | P = 1, T = 12 | Negative | Whipple | 3 months of gemsitabine/ |
| | | | | | | | | | | Abraxane + IMRT |
| HMI-024 | F | 82 | Head | Grade 2 | ypT3 | upN1 | P = 7, T = 29 | Negative | Whipple | 6 months gemsitabine/Abraxane |
| HMI-025 | F | 62 | Head | Grade 2 | pT3 | pN1 | P = 2, T = 23 | Focal positive | Whipple | No prior therapy |
| HMI-028 | M | 82 | Head | Grade 2-3 | pT3 | pN1 | P = 7, T = 15 | Negative | Whipple | No prior therapy |
| HMI-034 | M | 58 | Head | Grade 2 | ypT3 | ypN1 | P = 4, T = 22 | Negative | Whipple | 4 months FOLFIRINOX + SBRT |
| HMI-036 | M | 71 | Head | Grade 1 | pT3 | pN1 | P = 2, T = 25 | Negative | Whipple | No prior therapy |
| HMI-038 | M | 72 | Head | Grade 2 | pT3 | pN0 | P4, T = 31 | Negative | Whipple | No prior therapy |
| HMI-039 | M | 65 | | Grade 1 | | pN0 | p = 0, T = 11 | Negative | Pancreatectomy | No prior therapy |
| HMI-042 | M | 63 | | Grade 2-3 | | pTN1 | p = 1, T = 15 | Negative | Distal pancreatectomy | Gemcitabine for short time |
| HMI-043 | F | 71 | | Grade 1-2 | | PN1 | p = 2, T = 22 | Positive | Distal pancreatectomy | Chemo (GTX) |
| HMI-046 | F | 44 | | Grade 2 | | PN1 | p = 1, T = 11 | Positive | Pancreatectomy | Chemo (Gem) |
| HMI-047 | F | 80 | | Grade 2 | | pN1 | p = 4, T = 19 | Negative | Distal pancreatectomy | No prior therapy |
| HMI-050 | F | 65 | | Grade 2-3 | pT3 | pN1 | p = 8, T = 26 | Positive | Distal pancreatectomy | |
| HMI-051 | M | 70 | | Grade 3 | pT3 | pN1 | P = 3, T = 20 | Negative | Distal pancreatectomy | No prior therapy |
| HMI-052 | F | 54 | Head | Grade 3 | pT3 | pN1 | p = 3, T = 25 | Negative | Whipple | No prior therapy |
| HMI-053 | F | 68 | Head | | | | | | Whipple | FFX |
| HMI-055 | F | 64 | Head | Grade 2-3 | pT3 | pN0 | p = 0, T = 18 | Negative | Whipple | No prior therapy |
| HMI-056 | F | 77 | Head | Grade 2-3 | pT3 | pN1 | P = 1, T = 10 | Negative | Whipple | |
| HMI-057 | F | 76 | | Grade 2 | pT1 | pN0 | P = 0, T = 34 | Negative | Distal pancreatectomy | No prior therapy |
| HMI-058 | F | 53 | | Grade 1-2 | ypT3 | ypN1 | p = 4, T = 27 | Negative | Distal pancreatectomy | Chemo (FFX), SBRT |
| HMI-059 | F | 65 | Head | Grade 2-3 | ypT3 | ypN1 | p = 3, T = 19 | Positive | Whipple | FFX, SBRT |
| HMI-060 | M | 73 | Head | Grade 2-3 | pT3 | pN1 | p = 2, T = 17 | Negative | Whipple | |
| HMI-063 | M | 72 | | Grade 2 | ypT3 | ypN0 | p = 0, T = 17 | Negative | Distal pancreatectomy | Chemo (FFX) + SBRT |
| HMI-066 | F | 74 | Head | Grade 3 | pT2 | pN1 | p = 1, T = 14 | Negative | Whipple | FFX |

For the specimens in all categories, tumors, perilesional, and non-neoplastic (normal) areas surrounding the pancreatic tumor were identified because of the significant differences in their estimated Young's modulus. FIG. 8 illustrates the HME method application on surgical specimens with no chemotherapy and with neoadjuvant chemotherapy and radiotherapy. In addition, Mason's trichrome slide and Picrosirius red slide associated with various parts of the specimen: tumor, perilesional and non-neoplastic parts are shown. The Picrosirius red can quantify the fibrosis amount for each part of the specimens. Both the estimated corresponding Young's modulus of the specified part, along with the PSR density percentage is indicated. As these aforementioned results showed, the detected Young's modulus values for all of these cases were in accordance with Picrosirius red staining methodology as an independent microscopic method. In other words, elevation in collagen density rendered increasing the estimated Young's modulus measured with HME.

FIG. 8A shows a cross-section photograph of the PDA tumor and its surrounding tissue. FIG. 8B shows a B-mode image of the PDA tumor and its surrounding tissue. FIG. 8C shows a 2D Young's modulus map overlaid on the B-mode image. The estimated median Young's modulus for tumor part, red/orange/yellow part is (YM=44.9 kPa). The estimated median Young's modulus for a perilesional part, light blue part, is (YM=19.2 kPa). The estimated median Young's modulus for the non-neoplastic part, dark blue part is (YM=3.8 kPa). FIG. 8D shows a picrosirius red slide of the PDA tumor part with 20× magnification (PSR, density=53.2%). (e) Picrosirius red slide of perilesional part of PDA tumor surrounding with 20× magnification (PSR, density=35%) %). FIG. 8F shows a Picrosirius red slide of none-neoplastic, normal, part of PDA tumor surrounding with 20× magnification (PSR, density=4.6%). FIG. 8G shows Mason's trichrome slide with 20× magnification of the PDA tumor part. FIG. 8H shows Mason's trichrome slide with 20× magnification of adjuvant, perilesional, part of PDA tumor surrounding. FIG. 8I shows Mason's trichrome slide with 20× magnification of none-neoplastic, normal, part of PDA tumor surrounding. FIGS. 8J to 8R show a surgical pancreatic human specimen with chemotherapeutic history. (Gemcitabine/Abraxane, 6 months): (8J) Cross-section photograph of PDA tumor and its surrounding tissue. (8K) B-mode image of PDA and its surrounding tissue. (8L) 2D Young's modulus map overlaid on the B-mode image. The estimated median Young's modulus for tumor part, red/orange/yellow area, is (YM=35.3 kPa). The estimated median Young's modulus for a perilesional part, light blue part, is (YM=18 kPa). The estimated median Young's modulus for the non-neoplastic part, dark blue part is (YM=3.1 kPa). (8M) Picrosirius red slide of PDA tumor part with 20× magnification (PSR, density=47.1%). (8N) Picrosirius red slide of adjuvant, perilesional, part of PDA tumor surrounding with 20× magnification (PSR, density=35.4%). (8O) Picrosirius red slide of none-neoplastic, normal, part of PDA tumor surrounding with 20× magnification (PSR, density=6.6%). (8P) Mason's trichrome slide with 20× magnification of the PDA tumor part. (8Q) Mason's trichrome slide with 20× magnification of adjuvant, perilesional, part of PDA tumor surrounding. (8R) Mason's trichrome slide with 20× magnification of none-neoplastic, normal, part of PDA tumor surrounding. FIGS. 8S-8δ show a surgical pancreatic human specimen with chemotherapeutic history: (8S) cross-section photograph of PDA tumor and its surrounding tissue. (8T) B-mode image of PDA and its surrounding tissue. (8U) 2D Young's modulus map overlaid on the B-mode image. The estimated median Young's modulus for tumor part, red/orange/yellow part, is (YM=40 kPa). The estimated median Young's modulus for adjuvant, perilesional, part of PDA tumor surrounding, light blue area, is (YM=17.2 kPa). The estimated median Young's modulus for none-neoplastic, normal, part of PDA tumor surrounding, dark blue, is (YM=2.9 kPa). (8V) Picrosirius red slide of PDA tumor part with 20× magnification (PSR, density=57%). (8 W) Picrosirius red slide of adjuvant, perilesional, part of PDA tumor surrounding with 20× magnification (PSR, density=23.6%). (8X) Picrosirius red slide of none-neoplastic, normal, part of PDA tumor surrounding with 20× magnification (PSR, density=5%). (8Y) Mason's trichrome slide with 10× magnification of the PDA tumor part. (8Z) Mason's trichrome slide with 10× magnification of adjuvant, perilesional, part of PDA tumor surrounding. (8δ) Mason's trichrome slide with 10× magnification of none-neoplastic, normal, part of PDA tumor surrounding.

In order to observe the relationship between measured stiffness, YM, using the HME method and fibrosis progress, the Picrosirius red staining method was applied to tumor, perilesional, and non-neoplastic part of the post-surgical human specimens. The result is illustrated in FIG. 9(d). Table 3 demonstrates those results with more details.

TABLE 3
Summary of applying both HME and Picrosirius
red method on pancreatic human

| | Number of | Young's modulus | PSR |
|---|---|---|---|
| Region | specimens | (HME), kPa | density (%) |
| None- | 7 | 3.5 ± 0.5 | 5.3 ± 1.1 |
| neoplastic, N | | | |
| Perilesional, | 6 | 23.5 ± 5.6 | 26.1 ± 9.8 |
| P | | | |
| Tumor, T | 11 | 44.8 ± 5 | 51.4 ± 23.3 |

The development of extremely fibrotic stroma in PDA tumors is due to the dense and cross-linked extracellular matrix (ECM) formation that occurs as the disease progresses. Fibrosis promotes stiffness elevation in the stroma, and the newly developed rigid stroma promotes tensional homeostasis that culminates in a high level of cell contractility. The performance of the HME method in assessing pancreatic tumors in transgenic mice with various levels of fibrosis was evaluated using the disclosed subject matter. The results showed a strong correlation between the reported Young's modulus value using HME and the fibrosis level seen on histologic examination. The 2D peak-to-peak maps measured by HMI corroborated well with the 2D Young's modulus maps generated by HME.

FIG. 9 summarizes the mouse and surgical human specimen's findings, showing a strong correlation between measured Young's modulus using HME and collagen density using the microscopic PSR method. FIG. 9 demonstrates HME and measuring Young's modulus facilitated recognition of three different regions in these human specimens: non-neoplastic region, N, perilesional region, P, and tumor region, T. This capability of HME can assist surgical planning in delineating tumor boundaries intraoperatively. This can potentially reduce positive surgical margins, increase resection rates, and reduce recurrence rates for this aggressive neoplasm. FIG. 9A shows Young's modulus measured in vivo in the normal pancreas (n=18). YM=(4.2±1.3) kPa, in inflamed pancreas with less than 50% fibrosis (n=18). YM=(4.7±1.1) kPa, in inflamed pancreas with more than 50% fibrosis (n=8). YM=(7.2±2.3) kPa, and in pancreatic tumors (n=5). YM=(11.3±1.7) kPa. (One-Way ANOVA: **P<0.0025, ***P<0.0003, ****P<0.0001). FIG. 9B shows the estimated median of Young's modulus values vs. PSR density percentage using Picrosirius red staining. This method was applied on 5 of the total normal pancreas, PSR=(2±0.8) %, on 17 of the whole fibrosis cases, PSR=(9.8±3.4) %, and on 4 with PDA, PSR=(13.2±1.2) %. FIG. 9C shows Young's modulus estimation of all PDA post-surgical specimens (n=32) regardless of their history therapy (One-Way ANOVA). N: Non-neoplastic parts, with a mean and standard deviation of YM=(4±1.6) kPa. P: Perilesional part, YM=(23.9±8) kPa. T: Tumor part, YM=(42.9±10.2) kPa. FIG. 9D shows the estimated median Young's modulus values vs. PSR density (%) using Picrosirius red staining of post-surgical human specimens. The number of T: tumor samples using PSR analysis are n=11 with mean and standard deviation of (51.4±23.3) %, for perilesional samples, P, using PSR analysis, n=6 with mean and standard deviation of (26.1±9.8) %, and the number of none-neo plastic or normal samples, N, using PSR on them, n=7 with mean and standard deviation (5.3±1.1)%.

Application of two independent methods, PSR and HME, on both transgenic mice with different fibrosis levels and in human PDA specimens had great advantages. For instance, the range of stiffness change in the former group was much lower than in the latter one. This meant that both the collagen density, PSR density (%), and HME were sensitive enough to detect the stiffness alteration in the range of 4 kPa to 12 kPa and fibrosis rate, PSR %, in the range of 2% to 14%, respectively, table 1. In the human specimens, these contrasts were much greater for these measured values, table 3. Another advantage of the disclosed subject matter was that HME was applied to both ex vivo and in vivo cases. This meant that the complexity of the in vivo model had no negative effects on HME Young's modulus measurement. This happened due to HME characteristics like its generated AM frequency deformation in tissue and its constant harmonic nature.

There is a significant difference between the measured Young's modulus values of PDA tumors in transgenic mice (YM<14 kPa) and in human specimens (YM<60 kPa). The HME Young's modulus estimation of PDA tumors corroborated previous studies using different mechanical testing for Young's modulus estimation in transgenic mice. Similarly, the same trend was obtained in human cases. In mice studies, the PSR density percentage for normal pancreas was (2±0.8) %, for fibrotic cases (KC) was (9.8±3.4) %, and for cases with more full-fledged tumor (KPC), PSR was (13.2±1.2) %, (FIG. 9 (a, b) and table 1). In human specimens, the PSR was (5.3±1.1) % for non-neoplastic, perilesional was (26.1±9.8) %, and tumor (51.4±23.3) % (FIG. 9 (c, d), and table 3).

The human specimens included chemotherapy, radiotherapy, and non-treated tumors. The chemotherapy effect on the stiffness of PDA tumors compared to non-treated tumors was insignificant. At the center of such tumors, the YM is significantly higher than in the normal and perilesional part regardless of their therapy history. However, what is apparent is the increase in the perilesional area surrounding the PDA tumor, as it is illustrated in FIG. 8. This area is larger in FIGS. 8 (l) and 8 (u) compared to FIG. 8 (c).

In spite of all the aforementioned advantages of HME, in some of the generated 2D Young's modulus maps like FIGS. 8 (l) FIG. 8 (u), there is an overestimation of Young's modulus estimation due to reflection at the boundary of the specimens. Overestimation did not affect the reported Young's modulus value related to non-neoplastic regions.

This disclosed subject matter was used to show that increasing fibrosis in murine PDA tumors was associated with an elevation in both collagen density measured by the Picrosirius red method and Young's modulus or stiffness of tumors. A similar trend in each individual pancreatic human specimen was observed in a way that three different regions were identified. The tumor region with the highest collagen density, % PSR, while the lowest measured PSR value corresponded to the non-neoplastic region. In addition, the measured PSR values related to perilesional regions fell in the intermediate range. 2D Young's modulus maps generated by HME of each human specimen showed a similar trend. HME has the capability of generating 2D Young's modulus maps non-invasively. This ability can provide new avenues for detecting and staging PDA tumors based on the collagen content, assessing the tumor response to chemotherapy, and assessing resectability.

### Example 3: Feasibility of the In Vivo Young's Modulus Visualization of Pancreatic Ductal Adenocarcinoma During HIFU Ablation Using Harmonic Motion Elastography (HME) In Vivo

Harmonic motion elastography technique: The system includes an imaging transducer located confocally in the middle of the 93-element, FUS transducer (fc=4.5 MHz, and D=70 mm, Sonic Concepts Inc., Bothell Wash., USA). The imaging transducer was either a 64-element phased-array imaging probe (fc=2.5 MHz, P4-2, ATL/Philips, Bothell, Wash., USA) or a 104-element diagnostic transducer (fc=7.8 MHz, P12-5, ATL/Philips, Bothell, Wash., USA). The former one was used in phantoms, while the latter one in mice. The FUS transducer is driven by an AM sinusoidal signal. A dual-channel arbitrary waveform generator (AT33522A, Agilent Technologies Inc., Santa Clara, Calif., USA) generates this AM signal through a 50-dB power amplifier (325LA, E&I, Rochester, N.Y., USA).

The total acoustic power output of the FUS transducer was in the range of 6.4-8.6 W based on radiation force balance measurements. The oscillatory motion generated by the FUS transducer is estimated by the channel data acquired by the imaging transducer (Vantage, Verasonics, and Bothell, Wash., USA). To reconstruct each RF data frame, the acquired channel data matrix is multiplied by the reconstruction sparse matrix, and its product matrix is multiplied by another sparse matrix for scan conversion. The whole process is implemented in the Graphical Processing Unit (GPU).

During this process, the data are upsampled at either 80 MHz for a 64-element phased array or 125 MHz for a 104-element transducer. The axial displacements at the focal point are estimated by applying a 1D normalized cross-correlation on the reconstructed RF data. To generate the 2D Young's modulus map, the same HMI displacement data of each point are used. Although it is possible to observe the shear wave propagation of the HMI displacement cine loop, a complex field of shear waves is generated due to constructive and destructive interaction of forward and reflected shear waves. To extract the shear wave from such a complex field, a directional filter is used to separate the leftward from the rightward shear waves. Because this Spatio-temporal filter is capable of disassembling the generated complex wavefield into its components, traveling in various directions. The filter was designed in frequency space with the ability to choosing the portion of the wave in a certain direction. In addition, this filtering method helps in minimizing the standing wave. These advantages contribute considerably to the reconstruction of the shear modulus of the medium.

The final Young's modulus 2D map is the result of applying this filter on HMI displacement data and using the time-of-flight algorithm to measure the time delay of the shear wave propagation by cross-correlating the filtered particle displacement profiles along the lateral direction. Then, two points separated by eight and six ultrasound wavelengths, in the phantom and mouse model, respectively, at the same depth, are used to calculate the time that it takes the shear wave to travel between these two points. Then, based on the estimated time delay between these points at a known distance, the shear wave speed is measured. The measured shear wave is assigned for the center pixel of the grid. The 2D shear wave map is generated for each HMI measurement, and the final 2D Young's modulus is reconstructed based on that.

Tissue-mimicking phantom model: A customized CIRS phantom (Model 049 A) with a cylindrical lesion of 5 mm diameter was used. The Young's modulus for the background and inclusion part in the phantom with the stiffer inclusion was 5±1, and 40±8 kPa, respectively. In the second phantom with a softer inclusion, Young's modulus of the inclusion was 10±2 kPa, and its background Young's modulus is the same as the stiffer phantom, 5±1 kPa.

In this phantom model, the radiation force was applied for 0.6 s using an amplitude-modulated waveform with an acoustic intensity of 1050 W/cm² and a frequency of 25 Hz, resulting in an excitation frequency of f=50 Hz. The imaging probe recorded plane waves at 1000 frames/s throughout the force application. It repeated five times by relocating the CIRS phantoms and changing the probe positions. The relative error based on

$$Erorr ( \% ) = \frac{{E}_{F} - {E}_{H}}{{E}_{F}} * 100 \qquad ( 2 )$$

where EF is Young's modulus of the CIRS phantom, and EH is Young's modulus measured by the HME method.
In addition, the contrast-to-noise ratio (CNR) was estimated based on

$$CNR ( dB ) = 20 log \frac{2 {( {E}_{i} - {E}_{b} )}^{2}}{{σ}_{i}^{2} + {σ}_{b}^{2}} , \qquad ( 3 )$$

where Ei and Eb are the mean Young's modulus of the inclusion and background, respectively; the ri and rb are the standard deviations of Young's modulus of the inclusion and background.

In vivo ablation experiment on transgenic mice with PDA tumor: The developed pancreatic tumor in these mice models (K-rasLSL.G12D/+; p53R172H/+; PdxCre (KPC)) were pathophysiologically similar to a human pancreatic tumor. Before starting the ablation, these KPC mice were abdominally depilated and laid supine on a heating pad under isoflurane anesthesia and covered with ultrasound gel. First, an 18.5-MHz diagnostic probe (L22-14v, Verasonics, Bothell, Wash., USA) was mounted on a 3D positioner and used to locate the pancreas and its surrounding organs due to its high-resolution B-mode image. In order to align the HMI images with high-resolution B-mode images, they were spatially registered with the high-resolution B-mode image by replacing this L22-14v transducer with the 104-element phased array (fc=7.8 MHz, P12-5, ATL/Phi-lips, Bothell, Wash., USA) without any change in the animal setting. This transducer was used for acquiring the frames while using HME. The FUS transducer was active for more than 60 s during the treatment at the same acoustic power (FIG. 1).

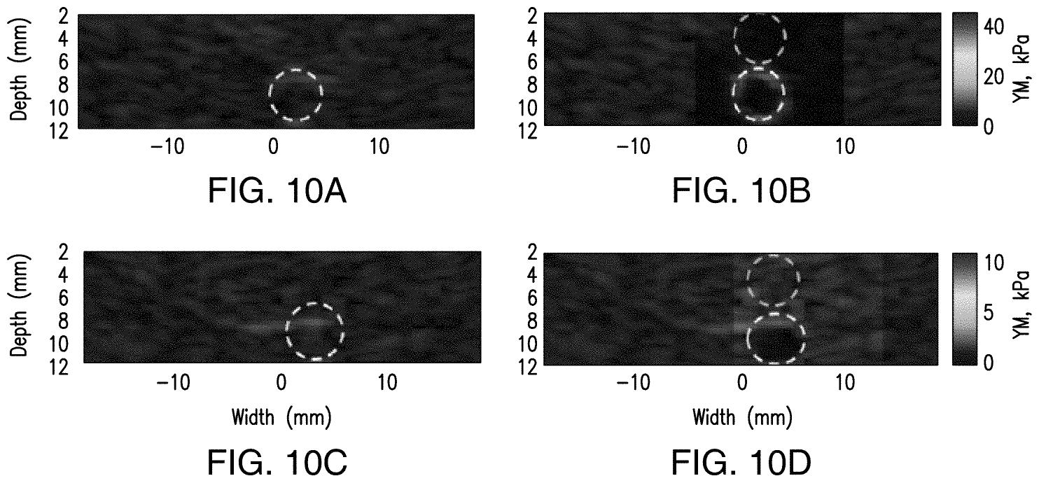

Phantom Model: In order to validate the HME methods, two modified CIRS phantoms (Model 049 A) with the cylindrical lesion of 5 mm diameter were used. FIG. 10 demonstrates the 2D Young's modulus reconstructed maps of these two phantoms using the HME method. The results are shown in Table 4. According to Table 4, the overall, largest error for the inclusion and background part is under 19%.

TABLE 4
Young's modulus, values, and contrast-to-noise ratio
(CNR) in CIRS phantom with stiffer inclusion.

| | | Back- | Error, | Error, | |
|---|---|---|---|---|---|
| | Inclusion | ground | inclusion | background | CNR |
| Trail | (Ei) | (Eb) | (%) | (%) | (dB) |
| 1 | 41.5 ± 9.3 | 4.8 ± 0.9 | 4 | 4 | 31.8 |
| 2 | 44.0 ± 10.8 | 4.7 ± 0.8 | 10 | 6 | 30.3 |
| 3 | 32.5 ± 6.5 | 4.7 ± 0.9 | 19 | 6 | 33.6 |
| 4 | 36.8 ± 10.6 | 4.7 ± 0.7 | 8 | 6 | 27.5 |
| 5 | 36.6 ± 11.9 | 4.3 ± 0.3 | 8 | 14 | 25.4 |
| AV | 38.3 ± 9.8 | 4.6 ± 0.7 | 10 | 8 | 29.5 |

FIG. 10A shows a B-mode image of the phantom with stiff inclusion. FIG. 10B shows an overlaid image of reconstructed 2D Young's modulus map on original B-mode in phantom with a stiff inclusion 1001. The estimated E for the background part specified with the dashed circle 1002 is 4.8±0.9 kPa. The dashed circle 1003 shows the lesion part and E=41.5±9.8 kPa. FIG. 10C shows a B-mode image of the phantom with soft inclusion 1004. FIG. 10D shows an overlaid image of reconstructed 2D Young's modulus map on original B-mode in phantom with a stiff inclusion. The estimated E for the background part specified with a dashed circle 1005 is 4.3±0.3 kPa. The dashed white circle shows the lesion part 1006, and E is about 10.1 kPa.

In addition, the CNR was higher than 25.4 dB. In the second CIRS phantom, the relative error for both the inclusion and background was less than 10%.

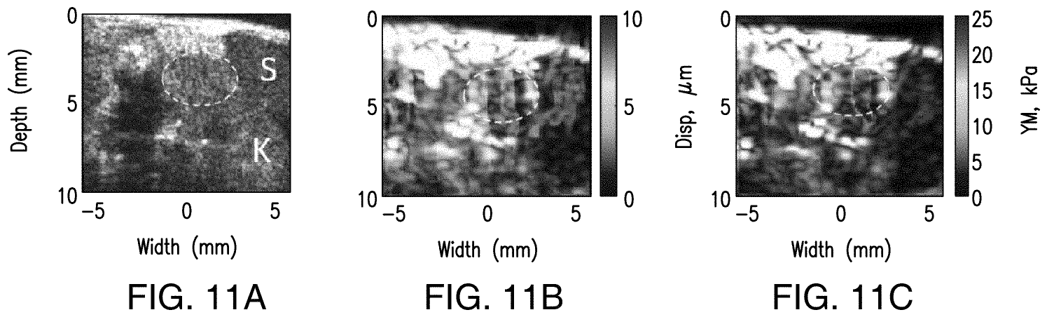

In vivo tumor models: FIG. 11A shows the high-resolution B-mode image of the pancreatic tumor, which is specified with a dashed oval 1101 and its surrounding organs. In this figure, the spleen and kidney are labeled with S and K, respectively. The ablation was performed on this tumor. At the end of the ablation, the resulting 2D absolute peak-to-peak displacement map and its corresponding 2D Young's modulus map are shown in FIG. 11B and FIG. 11C, respectively.

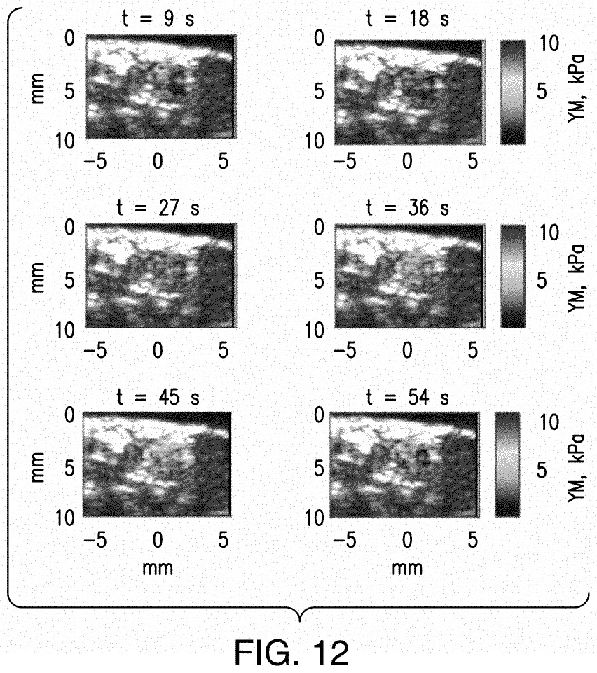

2D maps shown in FIG. 11 were reconstructed at the end of the HIFU application (after 57 s) in the first mouse. However, in order to monitor the stiffness changes occurring while performing the HIFU ablation method, the resulting 2D Young's modulus map was reconstructed every 9 seconds during HIFU ablation. The reconstructed 2D Young's modulus overlaid on the B-mode images is illustrated in FIG. 12.

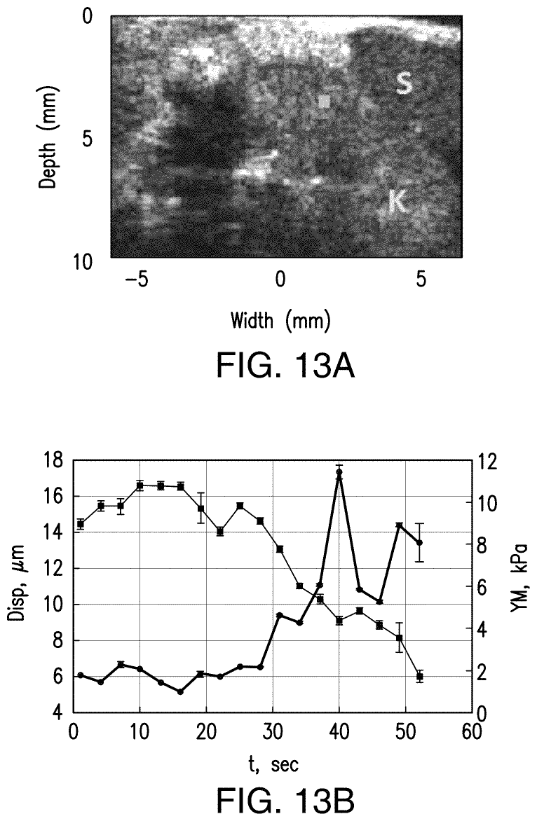

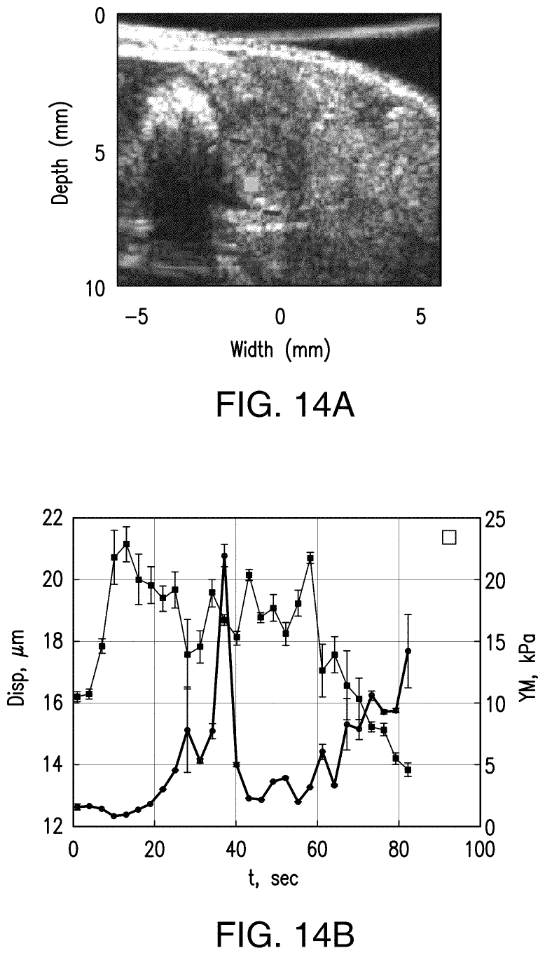

Moreover, in FIGS. 13 and 14, the temporal profile of the whole tumor specified with dashed oval shapes is demonstrated based on both absolute peak-to-peak displacement and Young's modulus estimation in the first and second in vivo mouse models, respectively.

FIG. 13A shows a high-resolution B mode image of PDA Tumor and surrounding organs of the first in vivo mouse study using L 22-14 V probe. The tumor is specified with a dashed red oval shape. FIG. 13B shows the absolute peak-to-peak displacement 1301 and Young's modulus temporal profiles 1302 of the tumor, dashed oval 1303, in part (13A) of this figure during HIFU application for 54 s. FIG. 14A shows a high-resolution B-mode image of PDA tumor and surrounding organs of the second in vivo mouse study using L22-14V probe. The tumor is specified with a dashed oval shape. FIG. 14B shows the absolute peak-to-peak displacement 1401 and Young's modulus temporal profiles 1402 of the tumor, dashed oval, 1403 in part (14A) of this figure during HIFU application for 84 s.

In both figures, the absolute peak-to-peak displacement and Young's modulus changes are in good agreement. The softer part shows higher displacement and lower Young's mechanical evaluation of soft tissues are becoming increasingly widespread due to their ease-of-use and ability to provide a quantitative 2D map of mechanical properties. Shear waves propagate faster in stiffer homogenous tissues comparing to softer ones. This difference in speed can be used to distinguish between normal and abnormal parts in tissue.

Shear wave attenuation can generate some artifacts in measuring shear wave speed. Using harmonic radiation force at a low frequency, 25 Hz, to generate shear wave can address the (SNR), especially in vivo and deep-seated organs, while attenuation problems pose a formidable challenge for other ultrasonic shear wave methods.

Furthermore, the use of a FUS transducer instead of an imaging one for radiation force application or FUS-push has a substantial impact on overcoming those limitations. Also, when it comes to lateral propagation, the estimation errors increase by propagating away from the perturbation region due to the lack of sufficient displacement. In addition, in HME, there is inherent registration between perturbation and the detection parts and no concern about damaging the probe because of high power application.

Due to the geometry of the FUS transducer, the resulting focal point is more focused. This characteristic helps to generate waves in all directions symmetrically. Moreover, the cylindrical symmetry of the shear wavefront can partially assist in lowering the attenuation effect and increasing the accuracy of the shear modulus and Young's modulus estimation of the medium. Also, it should be noted that the measured 2D Young's modulus map in the HME method is completely independent of the magnitude of applied radiation force or, consequently, the resulted absolute peak-to-peak displacement measurement. The disclosed HME technique can estimate Young's modulus of the tissue under ablation by measuring the speed of the resulting shear wave. Instead of having a transient push, which can be occurred among the aforementioned ultrasonic shear wave methods because of using the imaging transducer for that purpose, in the HME method, a continuous push is applied separately by a FUS transducer while the RF signal is recorded by imaging transducer. This combination helps to overcome those limitations.

The HIFU is used to generate ablation and/or the shear wave and, subsequently, 2D Young's modulus map.

The displacement and Young's modulus monitoring of in vivo tumor ablation confirm that a progressive softening-then-stiffening indicated by displacement increase-then-decrease and Young's modulus decrease-then-increase occurred (FIGS. 13 and 14). This trend happens not only for the tumor part. Almost similar changes occur in the background part, too, as shown in FIG. 12.

The results also show the softened area around the lesion boundary (FIG. 11). In addition, the speed of sound does not have any effects on displacement measurement in HMI and HME methods. Two different imaging methods: absolute peak-to-peak displacement (FIG. 11B) and HME (FIG. 11C), depict similar results.

In FIG. 11B, the peak-to-peak displacement value was not measured for the background part because there was no raster scanning involved, and the push was applied only in a single location at the center of the tumor. This is complementary information provided by HME, as it is shown in FIG. 11C. One push is enough to measure the stiffness of the medium, while in the conventional HMI method, raster scanning is necessary for stiffness evaluation of the medium. FIG. 12 demonstrates the reconstructed 2D maps every 9 s at the onset of the HIFU ablation process.

An ultrasonic shear wave-based technique using HMI was disclosed. The radiation force technique was capable of reconstructing the 2D Young's modulus of the tissue during and after ablation in vivo. The disclosed HME method is distinct from other shear wave methodology, as it uses oscillatory force that can separate motion from breathing and body movement as well as engage viscosity estimation.

### Example 4: Young's Modulus Mapping of the Ablated Region

A Focused Ultrasound (FUS) transducer induces an amplitude-modulated (AM) harmonic motion at the frequency of 50 Hz. An imaging probe aligned confocally with the FUS transducer acquires the resulted Radio Frequency (RF) signals simultaneously. To estimate the local induced displacement, a 1-D cross-correlation method is used. The shear wave can also be extracted at the same time by applying a 2D directional filter on the displacement. The 2D Young's modulus map is formed by measuring the shear wave speed. This new, fully quantitative imaging technique can be used to estimate the stiffness in tissues, especially during the HIFU ablation process. To test the feasibility of this new method, canine liver specimens were used. The 2D Young's modulus map of the pre-ablated liver was reconstructed. In the same fashion, 2D Young's modulus map of the post-ablated liver specimen was also generated. In addition, Young's modulus behavior of the ablated area during the HIFU ablation is demonstrated.

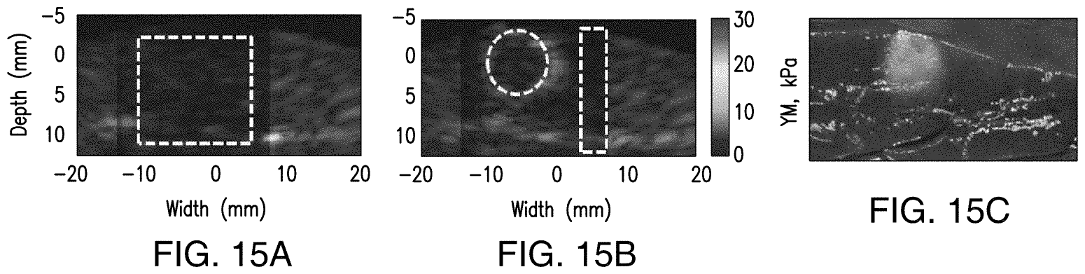

To validate the performance of this modulus imaging method, canine liver specimens were ablated for two minutes. FIG. 15A shows the overlay of the 2D Young's modulus map onto the compounding B-mode before ablation, while FIG. 15B represents the 2D Young's modulus map generated five minutes after the ablation process in this specimen. FIG. 15A shows a pre-ablated canine liver specimen. The estimated YM based on the white square area 1501 is (4.1±0.8) kPa. FIG. 15B shows a post-ablated liver specimen. The estimated YM for the ablated part specified as a white circle 1502 is (30.4±10.1) kPa, and for the background, part specified as white rectangular 1503 is (4.2±1) kPa. FIG. 15C shows a pathology image of the same liver specimen after HIFU exposure.

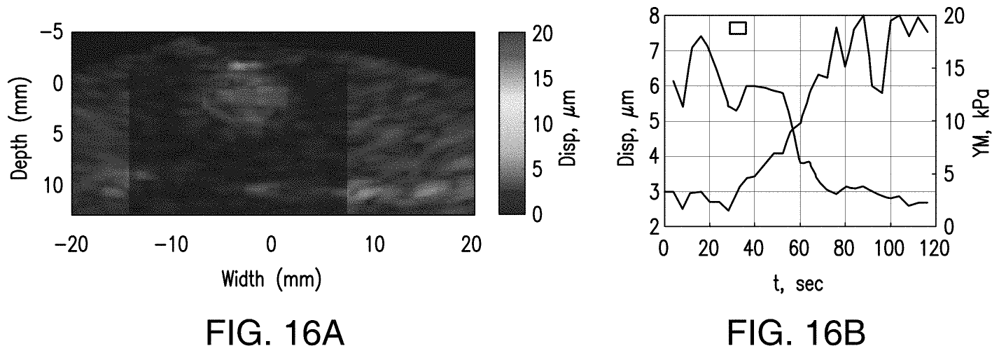

This modulus imaging can map Young's modulus not only after termination of HIFU ablation but also during the HIFU application. FIG. 16 illustrates Young's modulus behavior of the ablated area during the HIFU application. This figure shows that the ablated region becomes stiffer during the HIFU procedure after approximately 30 s of ablation and both temporal peak-to-peak displacement decrease and Young's modulus increase are in agreement regarding lesioning. FIG. 16A shows an overlay of the 2D displacement map after four seconds of HIFU application on its B-mode image of liver tissue. FIG. 16B shows Young's modulus profiles 1602 and displacement 1603 of the specified area 1601 (10×10) pixels in FIG. 16A during the HIFU application for 116 seconds.

The post-ablated region in the liver specimen was found to be approximately seven times stiffer compared to the pre-ablated one. In addition, both the peak-to-peak HMI displacement profile and Young's modulus estimation indicate the elevated stiffening during the HIFU procedure by reduction and increase, respectively, while Young's modulus map provided quantitative stiffness estimation.

The results presented herein demonstrate the capability of this new-HMI-based modulus imaging technique in measuring Young's modulus before, during, and after HIFU ablation.

### Example 5: Harmonic Motion Modulus Imaging

A Focused Ultrasound (FUS) transducer generates harmonic motion or pushes at an excitation frequency of 50 Hz. Simultaneously the imaging transducer aligned confocally with the FUS transducer acquires the radio frequency (RF) signals at a sampling rate of 1000 Hz. A 1-D cross-correlation method is applied to the data to estimate the local displacement. To extract the shear waves, a 2D directional filter is used on the displacement data. The 2D Young's modulus map is reconstructed by measuring the shear wave speed. To validate the HMMI estimates, a modified CIRS phantom (Model 049 A) with cylindrical lesion, 5 mm diameter, is used. The reported Young's modulus for the background and lesion was (5±1 kPa) and (40±8) kPa, respectively. After validation of the method, HMMI is applied to a human specimen with pancreatic ductal adenocarcinoma (PDA). In the phantom model, one push is used due to its small size, while for human specimens, multiple pushes are implemented to cover the whole specimen.

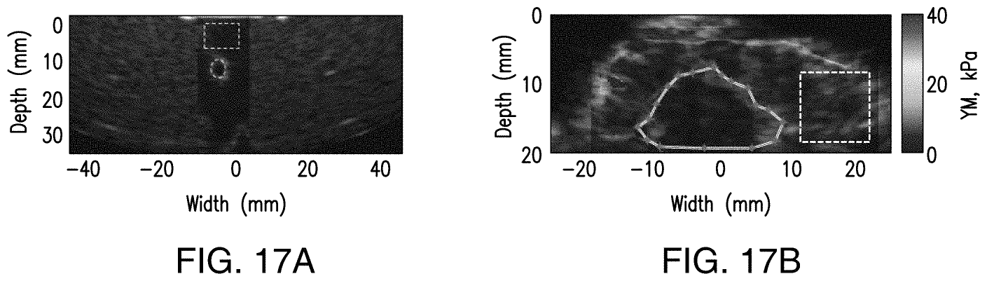

FIGS. 17A-17B show Young's modulus overlay on the B-mode image of both phantom and PDA in a human specimen. After repeating 5 times on phantom, the relative errors for lesion and background parts were 18% and 8%, respectively. Also, the contrast to noise ratio was found to be equal to 25 dB. The disclosed harmonic motion modulus imaging can accurately map Young's modulus of cancerous tissue. FIG. 17A shows an overlay image of reconstructed 2D Young's modulus map on original B-mode. White rectangular 1701 is the area specified for measuring the Young's modulus for the background part of CIRS phantom in which YM=(4.3±1.4) kPa. The dashed white circle 1702 shows the lesion part of CIRS phantom and YM=(36.6±6.6) kPa. FIG. 17B shows an overlay image of reconstructed 2D Young's modulus map on original B-mode. For lesion part 1703: YM is 42.7±16.7 kPa and for background 1704 YM is 4±1 kPa.

### Example 6: Harmonic Motion Elastography for Differentiation Between Pancreatic Ductal Adenocarcinoma from the Perilesional and Non-Cancerous Tissue in Post-Surgical Human Specimens

A Focused Ultrasound (FUS) transducer (4.5 MHz) generates harmonic motion at an excitation frequency of 50 Hz. A phased array (2.5 MHz) aligned confocally with the FUS transducer acquires the radio frequency (RF) signals at a frame rate of 1000 Hz. A 1-D cross-correlation method with a window size of 0.98 mm long and 95% overlap is applied on RF signals to estimate the local displacement. The shear wave speed can also be measured by applying a 2D directional filter on the displacement. The 2D Young's modulus (YM) value is measured based on the shear wave speed. To generate the final 2D Young's modulus map, a raster scan at 4 mm increments is performed in 2D. The translation of shear wave speed to Young's modulus is based on the assumption that soft tissue is incompressible, isotropic, linear, and elastic in which none of them are true in reality; however, it gives the closest estimation of stiffness using a noninvasive method like HME.

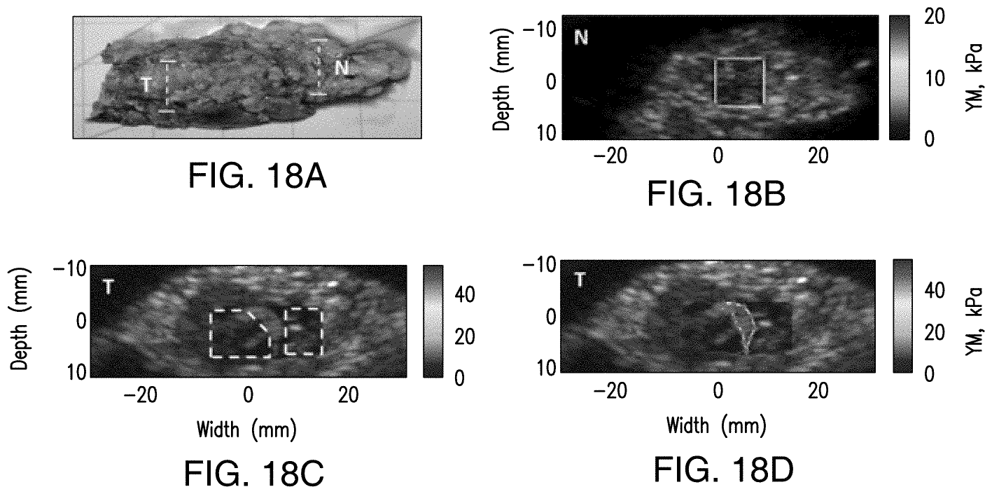

HME was applied on 19 specimens with PDA tumors. FIG. 18 depicts the 2D YM map using the HME method on one of the specimens. FIG. 18A shows a post-surgical pancreas specimen specified with T as the tumor part, and N is the non-cancerous part. FIG. 18B shows the overlay of 2D Young's modulus map of the non-cancerous part 1801 on its B-mode image. FIG. 18C shows the overlay 2D Young's modulus map of the tumor 1802 along with its perilesional region and non-cancerous part 1803 specified with the dashed line. FIG. 18D shows a perilesional part of the tumor specified by line 1804.

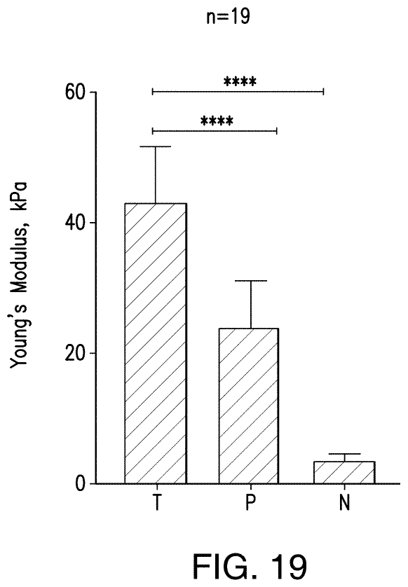

FIG. 19 illustrates the overall results of the HME application on 19 specimens. FIG. 19 shows the estimated Young's modulus for the tumor (Left) and perilesional tissue (Middle), and the non-cancerous region (Right). The results show that the disclosed HME has the capability of differentiating between the tumor, the perilesional tissue, and the non-cancerous region based on Young's modulus map ex vivo.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed is:

1. A system for harmonic motion elastography, comprising:
a focused ultrasound (FUS) transducer configured to generate an oscillatory motion of a target tissue by applying a push to the target tissue;
an imaging transducer configured to obtain radio frequency (RF) signals from the oscillatory motion during application of the push; and
a processor configured to estimate the mechanical properties of the target tissue by extracting a shear wave from the RF signals obtained using the imaging transducer and estimating a shear wave speed based on the extracted shear wave.

2. The system of claim 1, wherein the processor is configured to
conduct beamforming on the RF signals, and
generate a mechanical property map of the target tissue through a 1D cross-correlation.

3. The system of claim 1, wherein the mechanical properties comprises elasticity, stiffness, viscosity, poroelasticity, or combinations thereof.

4. The system of claim 1, wherein the processor is configured to generate a mechanical property map.

5. The system of claim 1, wherein the processor is implemented in a graphical Processing Unit (GPU).

6. The system of claim 1, wherein the push generates deformation of the target tissue.

7. The system of claim 4, wherein the system is configured to generate the mechanical property map with a single push.

8. The system of claim 1, wherein the FUS transducer is configured to move in a raster scanning manner.

9. The system of claim 1, wherein the processor is configured to identify a boundary between a lesion area and a non-lesion area.

10. The system of claim 1, wherein the imaging transducer is configured to obtain radio frequency data in real-time.

11. A method for measuring a mechanical property of target tissue, comprising:
generating an oscillatory motion of the target tissue by applying a push with a focused ultrasound (FUS) ultrasound transducer to the target tissue,
obtaining radio frequency (RF) signals from the oscillatory motion during application of the push to the target tissue using an imaging transducer;
extracting shear wave from the RF signals;
estimating shear wave speed from the extracted shear wave; and
estimating a mechanical property based on the RF signals.

12. The method of claim 11, further comprising
conducting beamforming on the RF signals, and
estimating RF displacement of the target tissue through a 1D cross-correlation.

13. The method of claim 11, further comprising moving the focused ultrasound in a raster scanning manner.

14. The method of claim 11, wherein the RF signals are obtained through a single push.

15. The method of claim 11, wherein the mechanical property comprises elasticity, stiffness, viscosity, poroelasticity, or combinations thereof.

16. The method of claim 11, further comprising adjusting a frequency of the push depending on the target tissue.

17. The method of claim 11, wherein the target tissue comprises a pancreatic ductal adenocarcinoma tumor.

18. The method of claim 11, further comprising identify a boundary between a lesion area and a non-lesion area of the target tissue.

19. The method of claim 12, further comprising extracting shear wave from the estimated RF displacement and estimating the shear wave speed.

20. The method of claim 19, further comprising calculating Young's modulus using the estimated shear wave speed and generating a Young's modulus map.
